(19) United States
(12) Patent Application Publication
(10) Pub. No.: US 2015/0194609 A1
(43) Pub. Date: Jul. 9, 2015

(54) DIBENZOXANTHENE COMPOUND, ORGANIC LIGHT-EMITTING DEVICE, DISPLAY, IMAGE INFORMATION PROCESSOR, AND IMAGE-FORMING APPARATUS

(71) Applicant: CANON KABUSHIKI KAISHA, Tokyo (JP)

(72) Inventors: Yosuke Nishide, Kawasaki-shi (JP); Naoki Yamada, Inagi-shi (JP); Akihito Saitoh, Gotemba-shi (JP)

(21) Appl. No.: 14/412,093

(22) PCT Filed: Jun. 24, 2013
(86) PCT No.: PCT/JP2013/067955

§ 371 Date: Dec. 30, 2014

(30) Foreign Application Priority Data
Jul. 3, 2012 (JP) 2012-149154

(51) Int. Cl. H01L 29/08; C07D 311/78; H05B 37/00

(52) U.S. Cl. CPC H01L 51/0058; C07D 311/78; H01L 51/0073; H01L 27/3244; H05B 33/0896; G03G 15/04036; H01L 51/5016

(57) ABSTRACT

Provided is a novel compound having a high lowest triplet excited level (T1 level), a narrow bandgap, and a shallow highest occupied molecular orbital (HOMO) level. A dibenzoxanthene compound is represented by formula [1] described in Claim 1. In formula [1], R₁ to R₇ are each independently selected from the group consisting of hydrogen, alkyl groups, aryl groups, heterocyclic groups, aryloxy groups, alkoxy groups, amino groups, silyl groups, and cyano groups.

### TECHNICAL FIELD

The present invention relates to dibenzoxanthene compounds, organic light-emitting devices containing such dibenzoxanthene compounds, and displays, image information processors, and image-forming apparatuses including such organic light-emitting devices.

### BACKGROUND ART

Organic light-emitting devices include a pair of electrodes and an organic compound layer disposed therebetween. Electrons and holes are injected from the pair of electrodes into the organic compound layer to cause a light-emitting organic compound contained therein to generate excitons, which emit light when returning to the ground state.

Organic light-emitting devices are also referred to as organic electroluminescent (EL) devices.

Recently, the utilization of phosphorescence has been proposed as an attempt to improve the luminous efficiency of organic EL devices. An organic EL device that utilizes phosphorescence is expected to provide an approximately four times higher luminous efficiency than an organic EL device that utilizes fluorescence.

PTL 1 discloses the following polymer and the following organic materials as materials for light-emitting layers in organic EL devices. The following polymer is referred to as “polymer 1,” and the following organic materials are referred to as “organic material a-1” and “organic material a-2.”

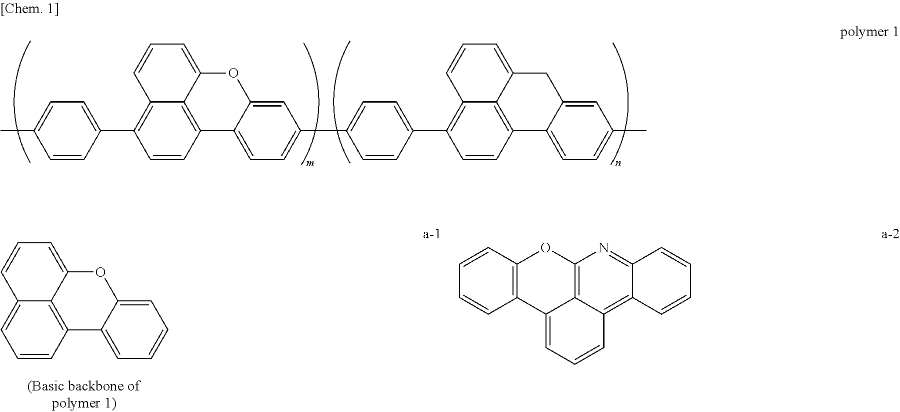

Organic material a-1, which is contained in polymer 1 disclosed in PTL 1, does not have a high lowest triplet excited level (T1 level). Organic material a-2, on the other hand, has a high T1 level but an excessively high lowest singlet excited level (S1 level).

### CITATION LIST

### Patent Literature

PTL 1 The specification of U.S. Patent Laid-Open No. 2009/0004485

### SUMMARY OF INVENTION

### Technical Problem

The present invention provides a dibenzoxanthene compound with a high T1 level and a narrow bandgap. The present invention also provides an organic light-emitting device, containing such a dibenzoxanthene compound, that has high luminous efficiency and that operates at low voltage, and a display, an image information processor, and an image-forming apparatus including such an organic light-emitting device.

### Solution to Problem

According to an aspect of the present invention, there is provided a dibenzoxanthene compound represented by general formula [1].

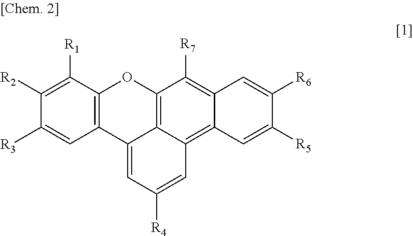

In the formula, R₁ to R₇ are each independently selected from the group consisting of hydrogen, substituted or unsubstituted alkyl groups, substituted or unsubstituted aryl groups, substituted or unsubstituted heterocyclic groups, substituted or unsubstituted aryloxy groups, substituted or unsubstituted alkoxy groups, substituted or unsubstituted amino groups, silyl groups, and cyano groups.

According to another aspect of the present invention, there is provided an organic light-emitting device including a pair of electrodes and at least one organic compound layer disposed between the pair of electrodes. The at least one organic compound layer contains the above dibenzoxanthene compound.

According to another aspect of the present invention, there is provided a display having a plurality of pixels. At least one of the plurality of pixels includes the above organic light-emitting device and an active device connected to the organic light-emitting device.

According to another aspect of the present invention, there is provided an image information processor including an input unit configured to receive image information and a display unit configured to display an image. The display unit is the above display.

According to another aspect of the present invention, there is provided a lighting apparatus including the above organic light-emitting device and an AC-DC converter circuit configured to supply a drive voltage to the organic light-emitting device.

According to another aspect of the present invention, there is provided an image-forming apparatus including a photoreceptor, a charging unit configured to charge a surface of the photoreceptor, an exposure unit configured to expose the photoreceptor to form an electrostatic latent image, and a developing unit configured to develop the electrostatic latent image formed on the surface of the photoreceptor. The exposure unit includes the above organic light-emitting device.

Further features of the present invention will become apparent from the following description of exemplary embodiments with reference to the attached drawings.

### BRIEF DESCRIPTION OF DRAWINGS

FIG. 1 is a schematic sectional view of an example of an organic light-emitting device including a stack of light-emitting layers.

FIG. 2 is a schematic sectional view of an example of a display including organic light-emitting devices according to an embodiment of the present invention and active devices connected to the organic light-emitting devices.

### DESCRIPTION OF EMBODIMENTS

An embodiment of the present invention relates to a dibenzoxanthene compound represented by general formula [1].

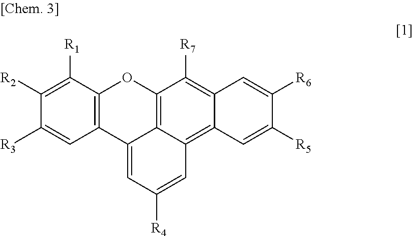

In general formula [1], R₁ to R₇ are each independently selected from the group consisting of hydrogen, substituted or unsubstituted alkyl groups, substituted or unsubstituted aryl groups, substituted or unsubstituted heterocyclic groups, substituted or unsubstituted aryloxy groups, substituted or unsubstituted alkoxy groups, substituted or unsubstituted amino groups, silyl groups, and cyano groups.

In this embodiment, examples of alkyl groups include alkyl groups having 1 to 4 carbon atoms, such as methyl, ethyl, n-propyl, isopropyl, n-butyl, isobutyl, sec-butyl, and tert-butyl.

In this embodiment, examples of aryl groups include phenyl, naphthyl, phenanthrenyl, fluorenyl, triphenylenyl, chrysenyl, and picenyl.

In this embodiment, examples of heterocyclic groups include pyridyl, oxazolyl, oxadiazolyl, thienyl, thiazolyl, thiadiazolyl, carbazolyl, acridinyl, and phenanthrolyl.

In this embodiment, examples of aryloxy groups include phenoxy and thienyloxy.

In this embodiment, examples of alkoxy groups include methoxy, ethoxy, propoxy, 2-ethyl-octyloxy, and benzyloxy.

In this embodiment, examples of amino groups include N-methylamino, N-ethylamino, N,N-dimethylamino, N,N-diethylamino, N-methyl-N-ethylamino, N-benzylamino, N-methyl-N-benzylamino, N,N-dibenzylamino, anilino, N,N-diphenylamino, N,N-dinaphthylamino, N,N-difluorenylamino, N-phenyl-N-tolylamino, N,N-ditolylamino, N-methyl-N-phenylamino, N,N-dianisolylamino, N-mesityl-N-phenylamino, N,N-dimesitylamino, N-phenyl-N-(4-tert-butylphenyl)amino, and N-phenyl-N-(4-trifluoromethylphenyl)amino.

In this embodiment, examples of silyl groups include triphenylsilyl.

In formula [1], the above substituents (i.e., the alkyl, aryl, heterocyclic, aryloxy, alkoxy, amino, and silyl groups) can be optionally further substituted with other substituents, including alkyl groups such as methyl, ethyl, propyl, and butyl; aralkyl groups such as benzyl; aryl groups such as phenyl, biphenyl, fluorenyl, and phenanthrenyl; heterocyclic groups such as pyridyl and pyrrolyl; amino groups such as dimethylamino, diphenylamino, and ditolylamino; and cyano groups.

In particular, R₁ to R₇ in formula [1] can each independently be selected from the group consisting of alkyl and aryl groups. A substituent composed only of a hydrocarbon forms a more stable compound than a substituent having a hetero atom.

The dibenzoxanthene compound according to this embodiment has the following properties:

(1) High T1 level

(2) Narrow bandgap

(3) Shallow highest occupied molecular orbital (HOMO) level

The dibenzoxanthene compound according to this embodiment, having the above properties, is suitable for organic light-emitting devices.

If the dibenzoxanthene compound according to this embodiment is used as a host material for a light-emitting layer of an organic light-emitting device, the organic light-emitting device can operate at low voltage because the dibenzoxanthene compound has superior charge injection properties.

In addition, the dibenzoxanthene compound according to this embodiment provides high luminous efficiency because efficient energy transfer occurs from the dibenzoxanthene compound to the guest material.

The dibenzoxanthene compound according to this embodiment is particularly effective for use as a host material for a light-emitting layer of an organic red phosphorescent device because the dibenzoxanthene compound has a T1 level suitable for red phosphorescent host materials.

As used herein, the term “red region” refers to the wavelength range of 550 to 680 nm.

A red phosphorescent guest material can have a T1 level of 550 to 680 nm. Accordingly, a host material can have a T1 level of less than 550 nm so that it has a higher T1 level than the guest material.

If the T1 level is expressed in terms of wavelength, a T1 level with a shorter wavelength has a higher energy. Hence, a T1 level with a wavelength of less than 550 nm has a higher energy than a T1 level with a wavelength of 550 nm.

The dibenzoxanthene compound according to this embodiment is suitable as a host material for red phosphorescent devices.

In this embodiment, the properties of the dibenzoxanthene compound are determined by molecular orbital calculations. The molecular orbital calculations are performed by the following quantum chemical calculations.

In this embodiment, the phrase “determined by calculations” means that the properties are determined by the following molecular orbital calculations.

In the molecular orbital calculations, the S1 level, the T1 level, the HOMO level, and the lowest unoccupied molecular orbital (LUMO) level are determined by the following technique.

The above molecular orbital calculations are performed by the density functional theory (DFT) method using the 6-31+G(d) basis functions in Gaussian 03 (Gaussian 03, Revision D.01, M. J. Frisch, G. W. Trucks, H. B. Schlegel, G. E. Scuseria, M. A. Robb, J. R. Cheeseman, J. A. Montgomery, Jr., T. Vreven, K. N. Kudin, J. C. Burant, J. M. Millam, S. S. Iyengar, J. Tomasi, V. Barone, B. Mennucci, M. Cossi, G. Scalmani, N. Rega, G. A. Petersson, H. Nakatsuji, M. Hada, M. Ehara, K. Toyota, R. Fukuda, J. Hasegawa, M. Ishida, T. Nakajima, Y. Honda, O. Kitao, H. Nakai, M. Klene, X. Li, J. E. Knox, H. P. Hratchian, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, P. Y. Ayala, K. Morokuma, G. A. Voth, P. Salvador, J. J. Dannenberg, V. G. Zakrzewski, S. Dapprich, A. D. Daniels, M. C. Strain, O. Farkas, D. K. Malick, A. D. Rabuck, K. Raghavachari, J. B. Foresman, J. V. Ortiz, Q. Cui, A. G. Baboul, S. Clifford, J. Cioslowski, B. B. Stefanov, G. Liu, A. Liashenko, P. Piskorz, I. Komaromi, R. L. Martin, D. J. Fox, T. Keith, M. A. Al-Laham, C. Y. Peng, A. Nanayakkara, M. Challacombe, P. M. W. Gill, B. Johnson, W. Chen, M. W. Wong, C. Gonzalez, and J. A. Pople, Gaussian, Inc., Wallingford Conn., 2004). This method is currently widely used.

Comparison of Dibenzoxanthene Backbone b-1 According to Embodiment with Comparative Compounds a-1 and a-2 Disclosed in PTL 1

Basic backbone b-1 of the dibenzoxanthene compound according to this embodiment is compared with comparative compound a-1, which is the basic backbone of polymer 1 disclosed in PTL 1.

The basic backbone of the dibenzoxanthene compound according to this embodiment is a backbone represented only by the fused ring structure in general formula [1]. That is, the basic backbone of the dibenzoxanthene compound according to this embodiment corresponds to the chemical structure of formula [1] where R₁ to R₇ are all hydrogen.

Comparative compound a-1 is represented by the following structural formula.

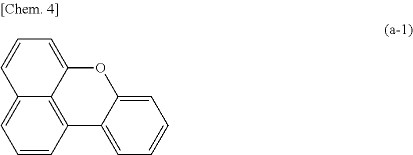 [SMILES: c1ccc2c(c1)Oc1cccc3cccc-2c13]

Comparative compound a-2 is represented by the following structural formula.

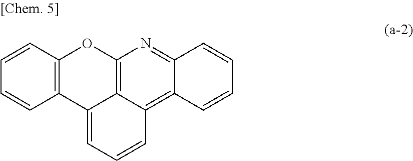 [SMILES: c1ccc2c(c1)Oc1nc3ccccc3c3cccc-2c13]

Dibenzoxanthene backbone b-1 according to this embodiment is represented by the following structural formula.

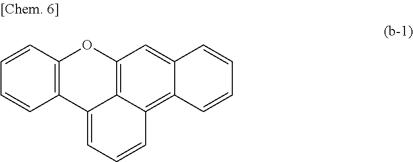 [SMILES: c1ccc2c(c1)Oc1cc3ccccc3c3cccc-2c13]

### Comparison for T1 Level

The T1 levels of the above compounds were calculated and compared. The T1 level of dibenzoxanthene backbone b-1 according to this embodiment was calculated to be 522 nm. The T1 level of comparative compound a-1 was calculated to be 544 nm. The T1 level of comparative compound a-2 was calculated to be 487 nm.

In particular, if the dibenzoxanthene compound according to this embodiment is used as a host material for an organic phosphorescent device, the dibenzoxanthene compound can have a higher T1 level than the guest material to allow efficient energy transfer to the guest material.

The T1 level of the light-emitting material that emitted red phosphorescence according to this embodiment was determined from the peak wavelength of an emission spectrum obtained in a dilute solution. The T1 level of the host material was determined from the rise in a phosphorescence spectrum obtained in a dilute solution.

The actual T1 level of dibenzoxanthene backbone b-1 according to this embodiment was 531 nm.

From the above results, the actual T1 level of comparative compound a-1 is estimated to be 550 nm or more. Thus, if comparative compound a-1 is used as a host material for an organic red phosphorescent device, it will not allow sufficient energy transfer to the guest material.

### Comparison for Bandgap

Next, the S1 levels of the above compounds were calculated. The S1 level of dibenzoxanthene backbone b-1 according to this embodiment was calculated to be 374 nm. The S1 level of comparative compound a-1 was calculated to be 362 nm. The S1 level of comparative compound a-2 was calculated to be 349 nm.

The S1 level was determined as the absorption edge wavelength obtained in a dilute solution, and the bandgap was determined from the S1 level.

The dibenzoxanthene compound according to this embodiment has a narrower bandgap than the comparative compounds.

### Comparison for HOMO Level

The HOMO levels of the above compounds were calculated. The HOMO level of dibenzoxanthene b-1 according to this embodiment was calculated to be −5.08 eV. The HOMO level of comparative compound a-1 was calculated to be −5.08 eV. The HOMO level of comparative compound a-2 was calculated to be −5.50 eV.

If a compound having a shallow HOMO level is used for an organic light-emitting device, the organic light-emitting device can operate at low voltage because the compound has a low charge injection barrier.

As used herein, the term “shallow HOMO level” refers to a HOMO energy level closer to the vacuum level.

That is, if the dibenzoxanthene compound according to this embodiment is used for an organic light-emitting device, it can operate at low voltage.

As described above, the above compounds were compared for properties (1) to (3). The results are shown in Table 1.

TABLE 1

| | Basic backbone | | |
|---|---|---|---|
| | b-1 | a-1 | a-2 |
| T1 level (nm) | 521 | 544 | 487 |
| S1 level (nm) | 374 | 362 | 349 |
| Difference between S1 and T1 levels (nm) | 147 | 182 | 138 |
| HOMO level (eV) | −5.08 | −5.08 | −5.50 |

Dibenzoxanthene backbone b-1 according to this embodiment has a small difference between S1 and T1 levels and a shallow HOMO level. These properties of dibenzoxanthene backbone b-1 are better than those of comparative compound a-1. In particular, the dibenzoxanthene compound according to this embodiment is more suitable for use as a red phosphorescent host material than comparative compound a-1 because comparative compound a-1 has a lower T1.

Comparative compound a-2 has a small difference between S1 and T1 levels, although it has a deeper HOMO level than dibenzoxanthene backbone b-1. Dibenzoxanthene backbone b-1, therefore, is more suitable as a host material for organic light-emitting devices than comparative compound a-2.

The shallow HOMO level of dibenzoxanthene backbone b-1 facilitates charge injection so that the organic light-emitting device can operate at low voltage.

The above comparisons demonstrate that dibenzoxanthene backbone b-1 has a high T1, a narrow bandgap, and a shallow HOMO level and is most suitable as a host material for organic light-emitting devices among the compounds compared.

Although dibenzoxanthene backbone b-1 according to this embodiment is compared in the above discussion, it will apply to all dibenzoxanthene compounds having dibenzoxanthene backbone b-1 according to this embodiment because the above properties are attributed to dibenzoxanthene backbone b-1.

Next, the effect of a substituent on the dibenzoxanthene compound according to this embodiment depending on the substituted position will be described.

Table 2 shows the calculated T1, S1, and HOMO levels of dibenzoxanthene compounds represented by general formula [1] where phenyl is attached at any of R₁ to R₇.

The S1 level, T1 level, HOMO level, LUMO level, and stability of the dibenzoxanthene compound according to this embodiment can be adjusted by attaching a substituent at any of R₁ to R₇ in general formula [1]. This effect varies depending on the substituted position.

For example, if phenyl is attached at any of R₁ to R₇ in general formula [1], the order of the T1 level is as follows: R₁>R₃, R₆>R₇>R₅>R₂, R₄. The T1 levels for R₂ and R₄, which are the lowest, are higher than the T1 levels of comparative compounds a-1 and a-2.

Thus, the dibenzoxanthene compound according to this embodiment has a high T1 level no matter where the substituent is attached.

The dibenzoxanthene compound according to this embodiment has a particularly high T1 level if the substituent is attached at R₁.

TABLE 2

| | Substituted position | | | | | | |
|---|---|---|---|---|---|---|---|
| | R₁ | R₃ | R₆ | R₇ | R₅ | R₂ | R₄ |
| Calculated T1 | 522 | 524 | 524 | 528 | 532 | 536 | 536 |
| level (nm) | | | | | | | |
| Calculated S1 | 380 | 379 | 378 | 380 | 382 | 393 | 381 |
| level (nm) | | | | | | | |
| Calculated HOMO | −5.08 | −5.07 | −5.10 | −5.03 | −5.04 | −5.06 | −5.07 |
| level (eV) | | | | | | | |

In general formula [1], the carbon atoms at the positions substituted with R₁ and R₇ have higher electron densities because of the influence of the adjacent oxygen atom. This means that the positions substituted with R₁ and R₇ are more reactive than other substituted positions.

Accordingly, the substituent can be attached at R₁ or R₇ to increase the chemical stability and the electrochemical stability. Thus, a dibenzoxanthene compound having the substituent at R₁ or R₇ has high chemical stability and electrochemical stability.

The dibenzoxanthene compound according to this embodiment requires no substituent having a high molecular weight to be attached to adjust the properties thereof because basic backbone b-1 itself has a high T1 level and a low S1 level (i.e., the basic backbone itself has suitable properties).

Because the dibenzoxanthene compound according to this embodiment requires no substituent having a high molecular weight, the entire molecule thereof has a low molecular weight. Thus, the dibenzoxanthene compound according to this embodiment is highly sublimable and can therefore be easily deposited by evaporation.

To improve the film-forming properties of the molecule, a substituent can be attached in at least one position. A substituent attached in one position does not significantly affect the sublimability.

Because the basic backbone of the dibenzoxanthene compound according to this embodiment has a low molecular weight, various types and numbers of substituents can be selected.

It is undesirable to attach a substituent having a high molecular weight to a basic backbone having a high molecular weight because the entire molecule thereof has a high molecular weight, which affects the properties of the resulting film. Thus, a basic backbone having a high molecular weight limits the range of substituents that can be selected.

Because the dibenzoxanthene backbone according to this embodiment has a wide range of substituents that can be selected, various properties thereof can be adjusted.

Examples of such properties include T1 level, S1 level, HOMO level, LUMO level, glass transition temperature, and sublimation temperature. A compound with a higher glass transition temperature provides better film-forming properties.

The dibenzoxanthene compound according to this embodiment has a shallow HOMO level, i.e., easily accepts holes. This is largely attributed to the electron-donating effect of the oxygen atom.

If the dibenzoxanthene compound according to this embodiment is used as a host material for an organic light-emitting device, its shallow HOMO level facilitates injection of holes into the light-emitting layer so that the organic light-emitting device can operate at low voltage.

The dibenzoxanthene compound according to this embodiment can be used as a material for organic light-emitting devices.

Examples of materials for organic light-emitting devices include materials used for hole-transporting layers, electron-blocking layers, light-emitting layers, hole-blocking layers, and electron-transporting layers.

For example, the dibenzoxanthene compound according to this embodiment can be used as a material for a light-emitting layer of an organic light-emitting device, particularly as a host material.

As used herein, the term “host material” refers to a compound that has the highest weight ratio of all the compounds forming a light-emitting layer. The term “guest material” is a compound that has a lower weight ratio than the host material among the compounds forming a light-emitting layer and that plays a major role in light emission. The guest material is also referred to as a dopant.

The term “assistant material” refers to a compound that has a lower weight ratio than the host material among the compounds forming a light-emitting layer and that assists the guest material. The assistant material is also referred to as a second host material.

Because the S1 and T1 levels of the dibenzoxanthene compound according to this embodiment are higher than the emission energy in the red region, it can be used as a material for red light-emitting devices.

The dibenzoxanthene compound according to this embodiment is not necessarily used as a host material for organic phosphorescent devices, but can also be used for hole-transporting layers and electron-transporting layers.

For example, the dibenzoxanthene compound according to this embodiment can be used as a hole-transporting material for an organic light-emitting device. Because of the electron-donating effect of the oxygen atom, the dibenzoxanthene backbone has a shallower HOMO level than a compound composed only of a hydrocarbon.

The shallow HOMO level of the dibenzoxanthene compound according to this embodiment facilitates injection of holes into the light-emitting layer so that the organic light-emitting device can operate at low voltage.

Table 3 shows the calculated HOMO levels of dibenzoxanthene backbone b-1 and comparative compounds, i.e., phenanthrene, chrysene, and triphenylene.

TABLE 3

| | b-1 | Phenanthrene | Chrysene | Triphenylene |
|---|---|---|---|---|
| Molecular structural formula | 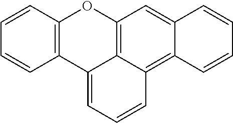 [SMILES: c1ccc2c(c1)Oc1cc3ccccc3c3cccc-2c13] | 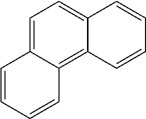 [SMILES: c1ccc2c(c1)ccc1ccccc12] | 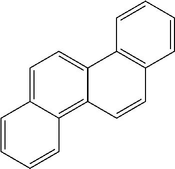 [SMILES: c1ccc2c(c1)ccc1c3ccccc3ccc21] | 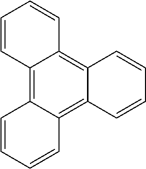 [SMILES: c1ccc2c(c1)c1ccccc1c1ccccc21] |
| Calculated | −5.08 | −5.73 | −5.51 | -5.85 |
| HOMO | | | | |
| level (eV) | | | | |

The dibenzoxanthene compound according to this embodiment can also be used for electron-transporting layers. In particular, a dibenzoxanthene backbone substituted with an aryl group is suitable for electron-transporting layers. Aryl groups allow electrons to be efficiently transported to the light-emitting layer because of their high electron-transporting ability.

The dibenzoxanthene compound according to this embodiment is not necessarily used for organic phosphorescent devices, but can also be used for organic fluorescent devices. For example, the dibenzoxanthene compound according to this embodiment can be used as a host material or for a hole-transporting layer or electron-transporting layer of an organic fluorescent device.

### Examples of Organic Compounds According to Embodiment

The dibenzoxanthene compound according to this embodiment is illustrated by the following non-limiting examples.

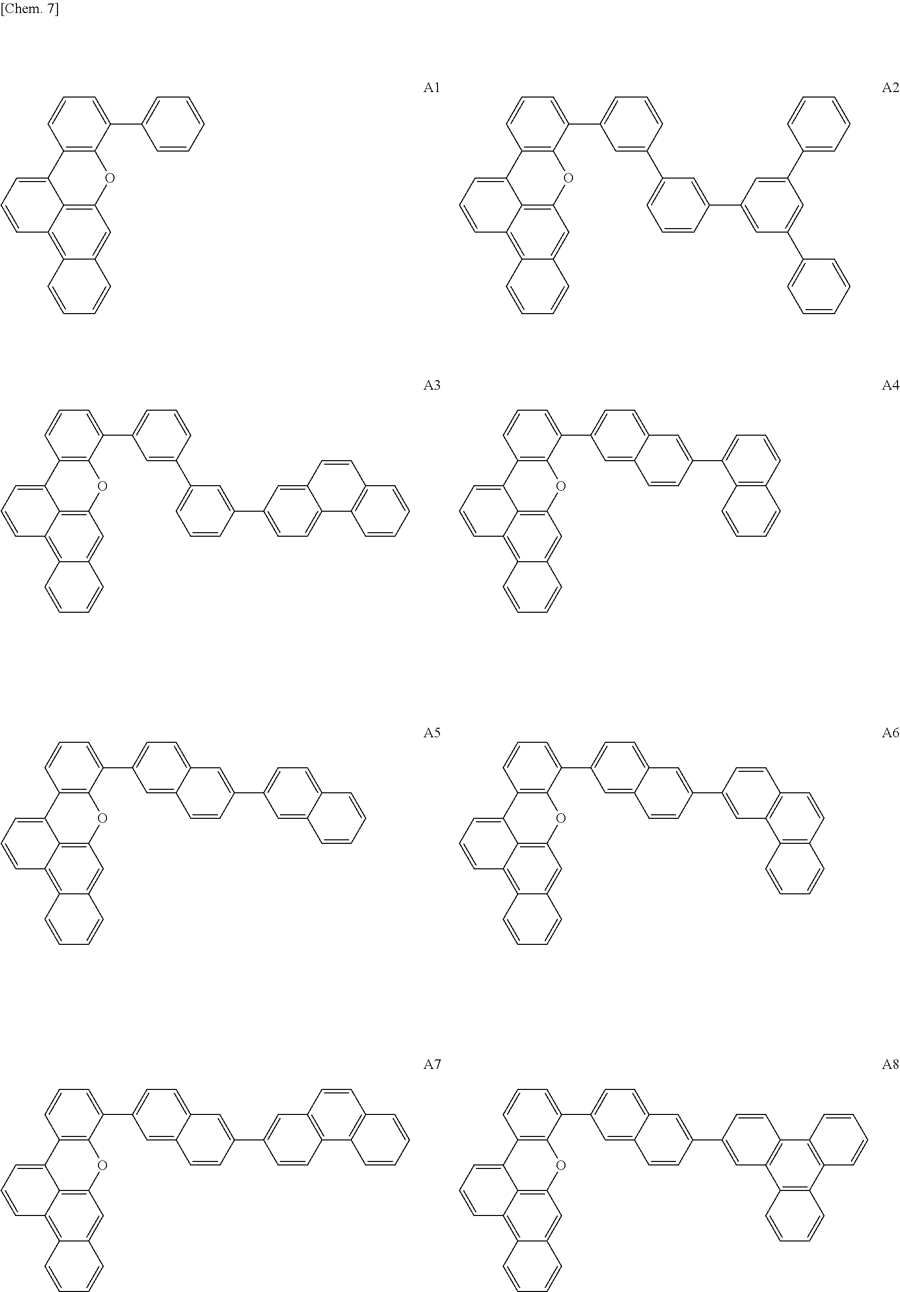

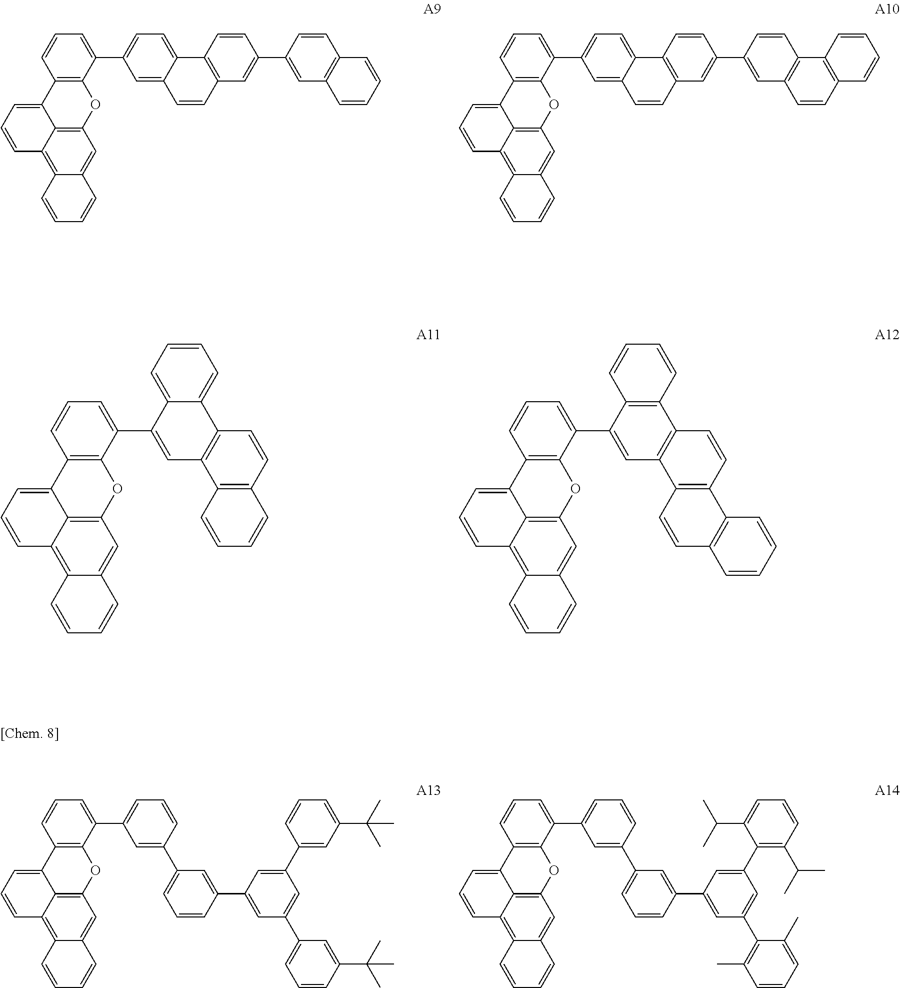

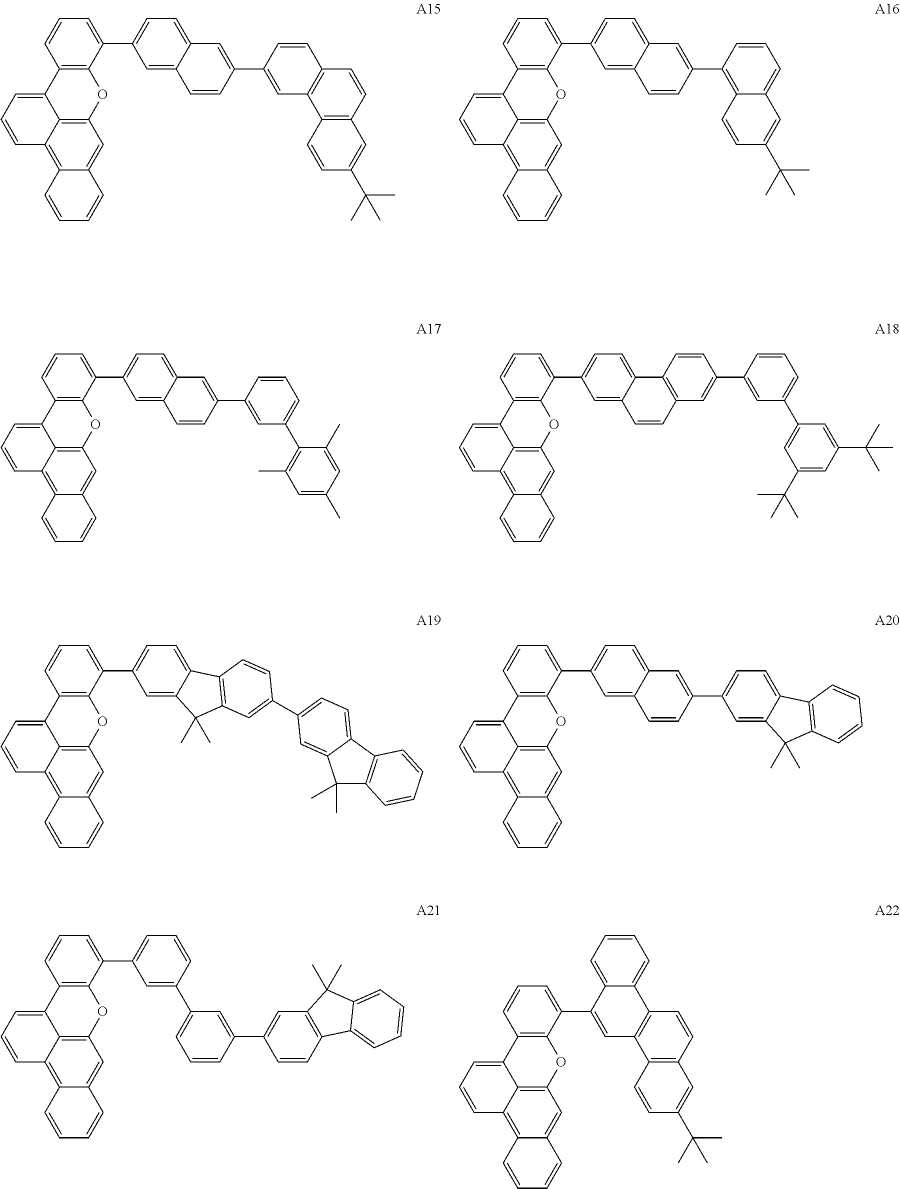

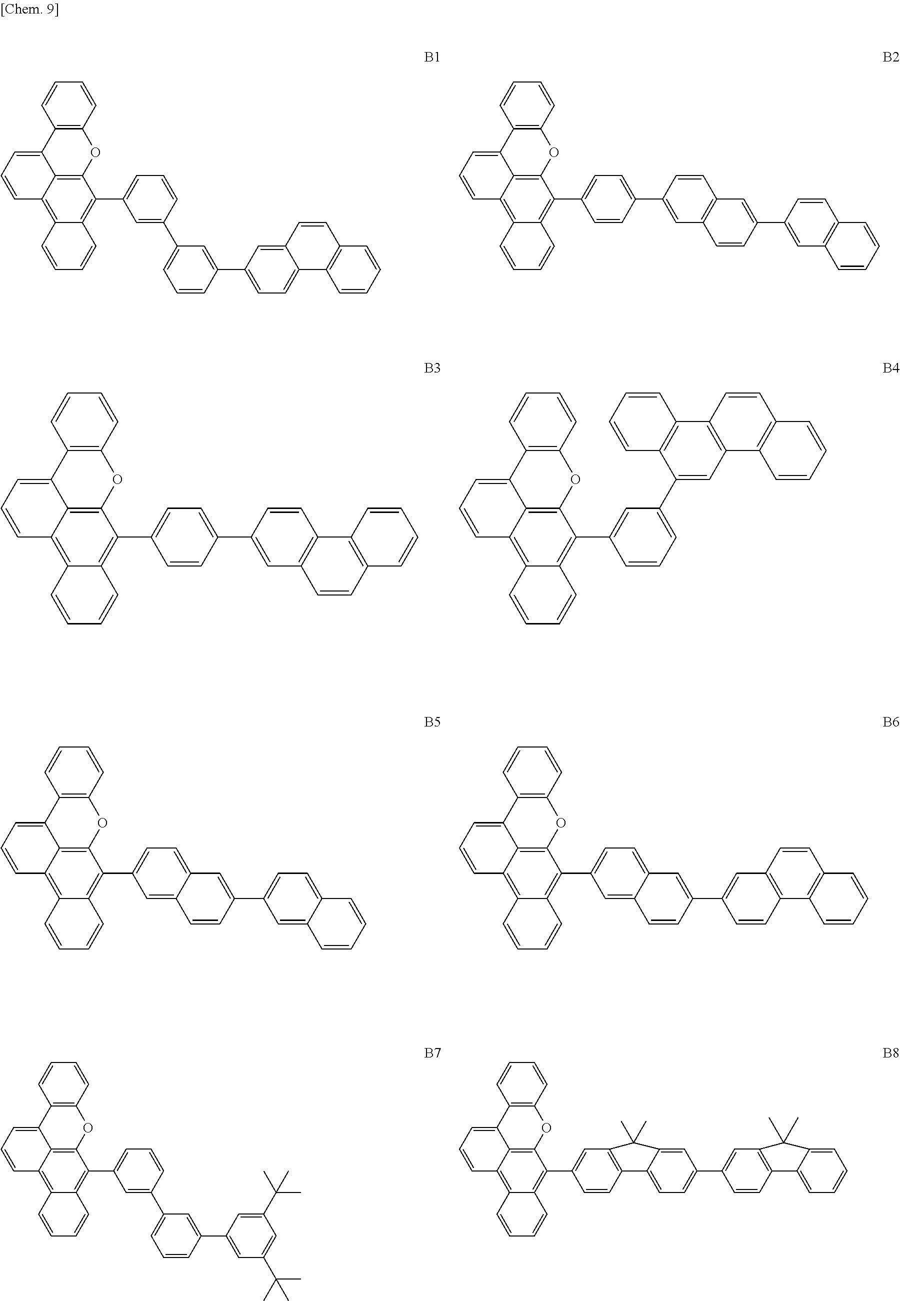

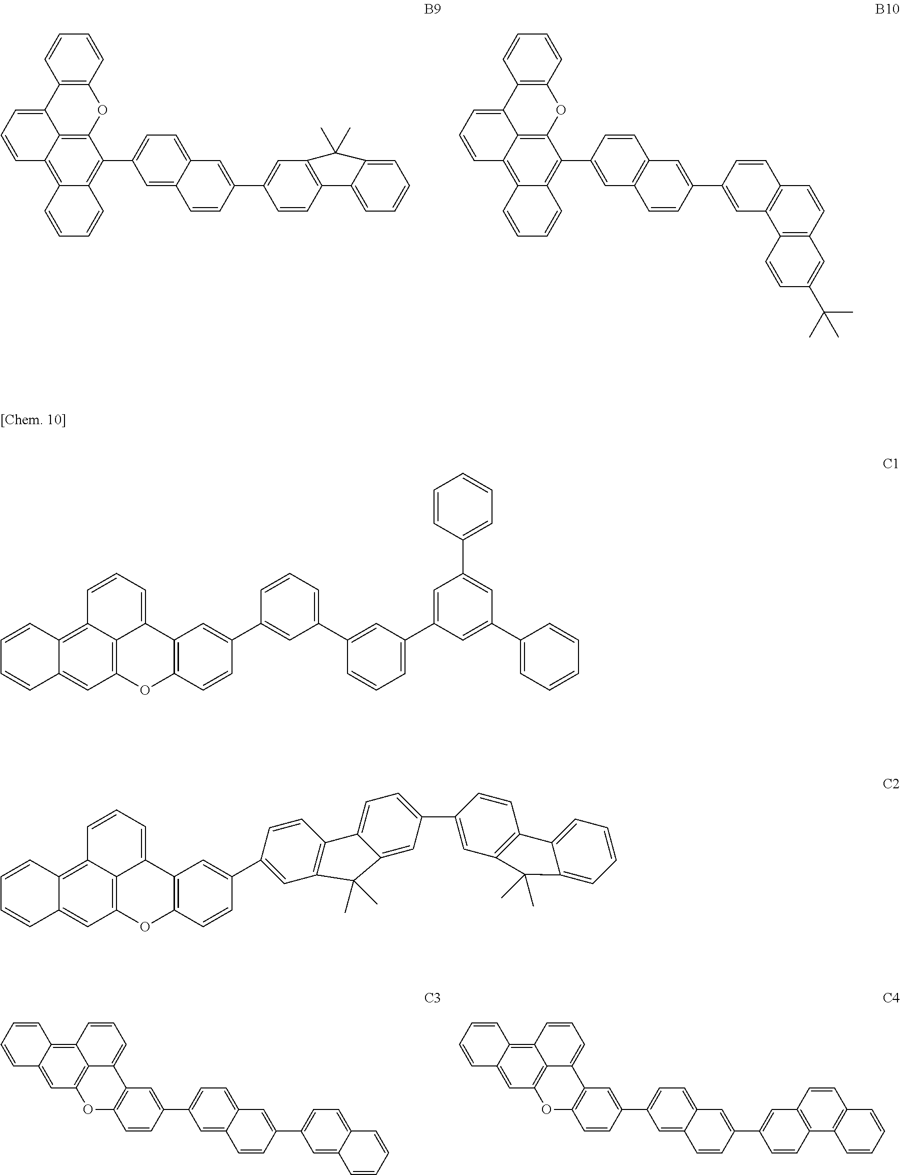

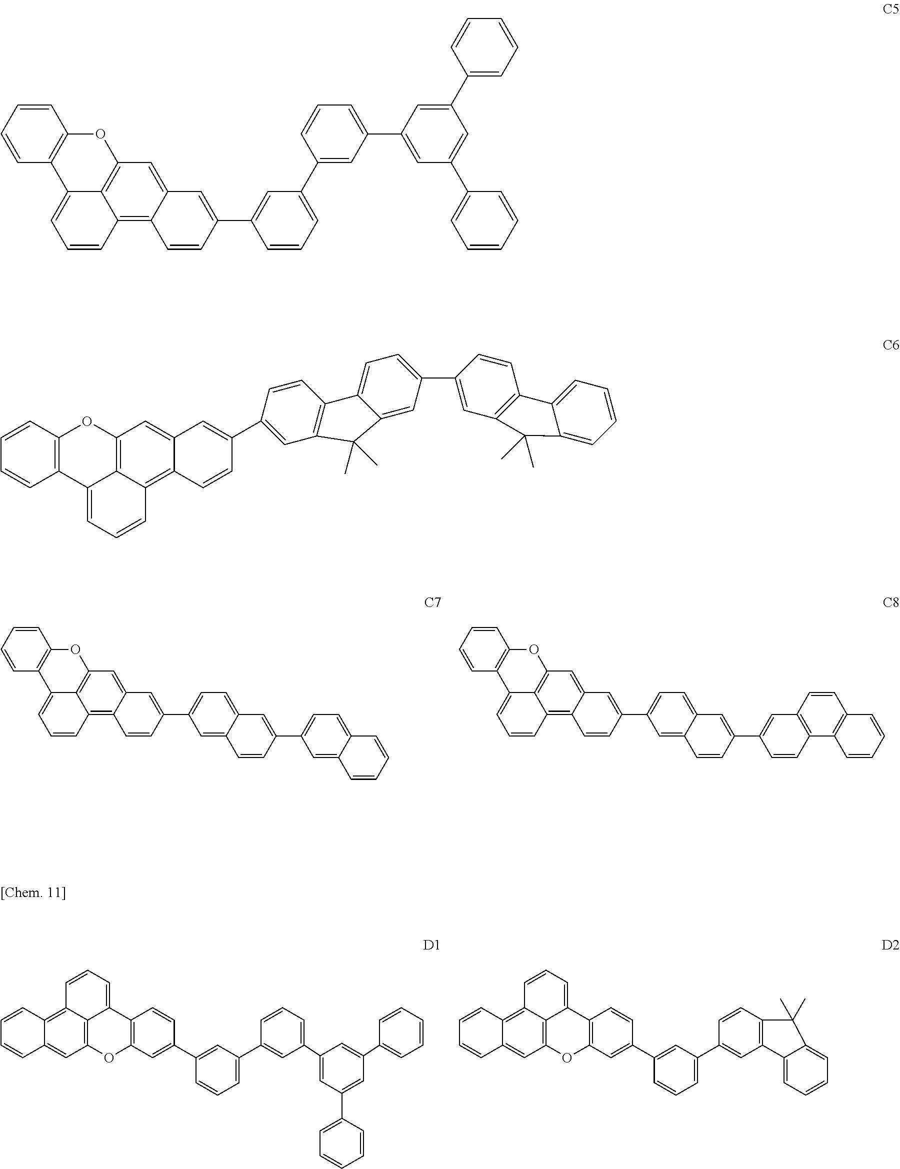

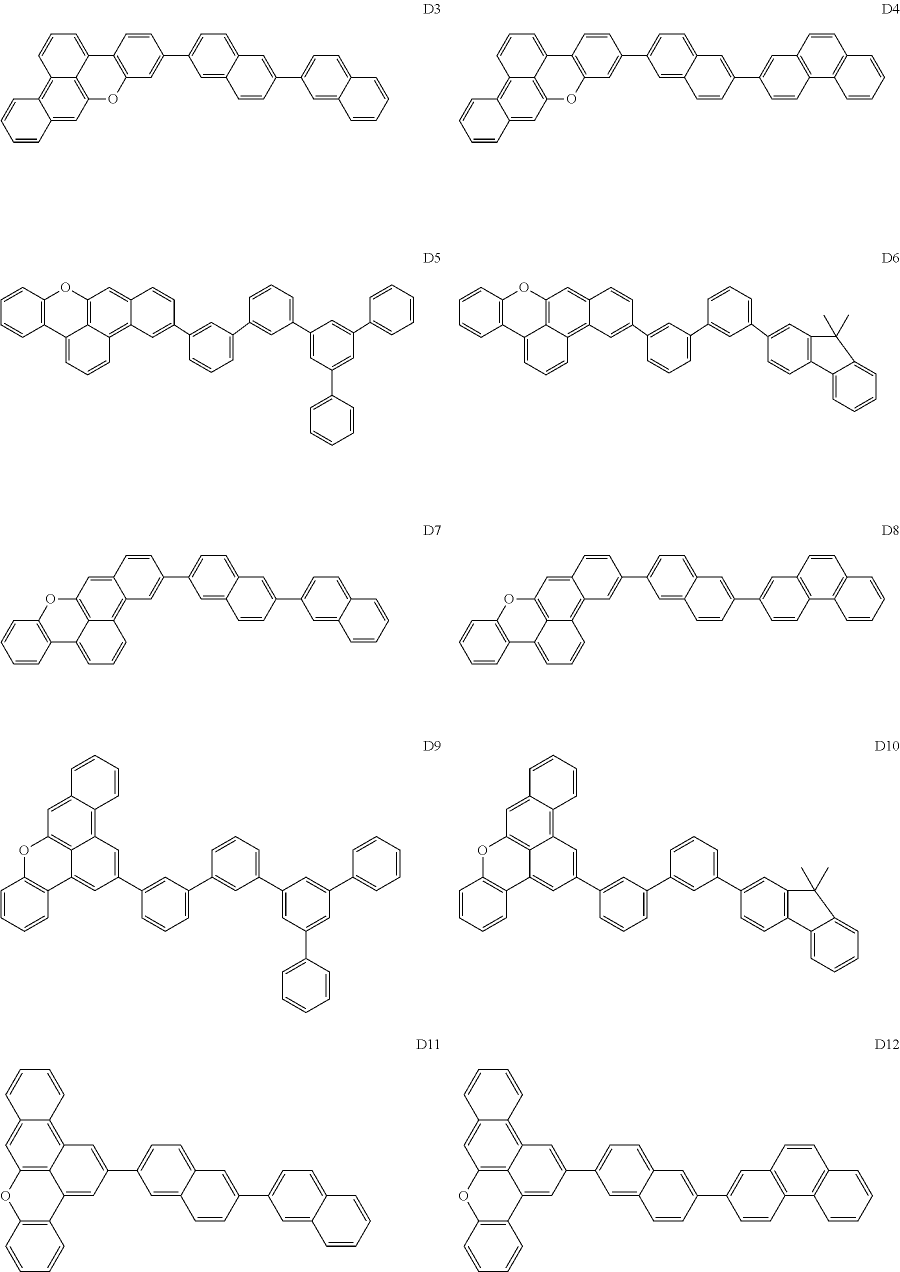

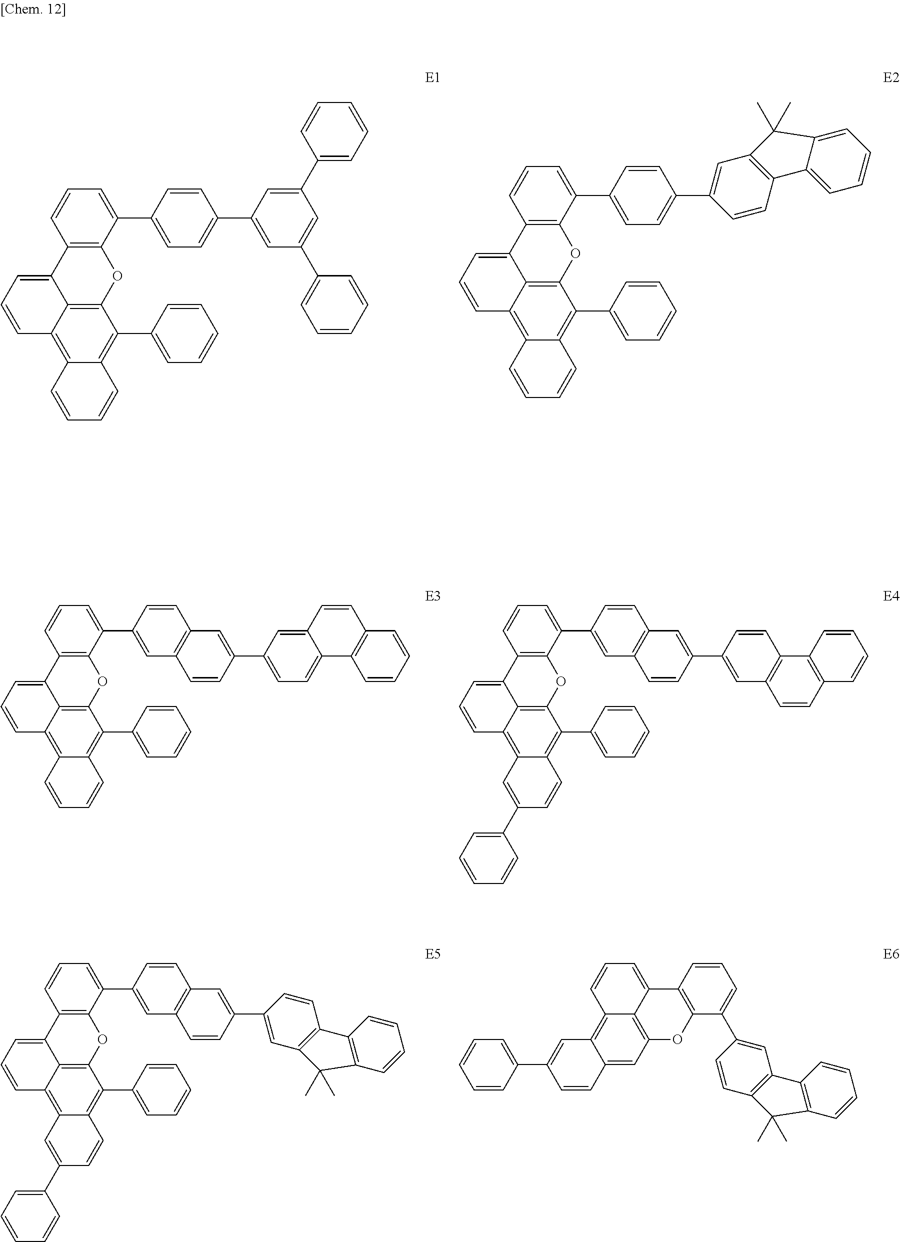

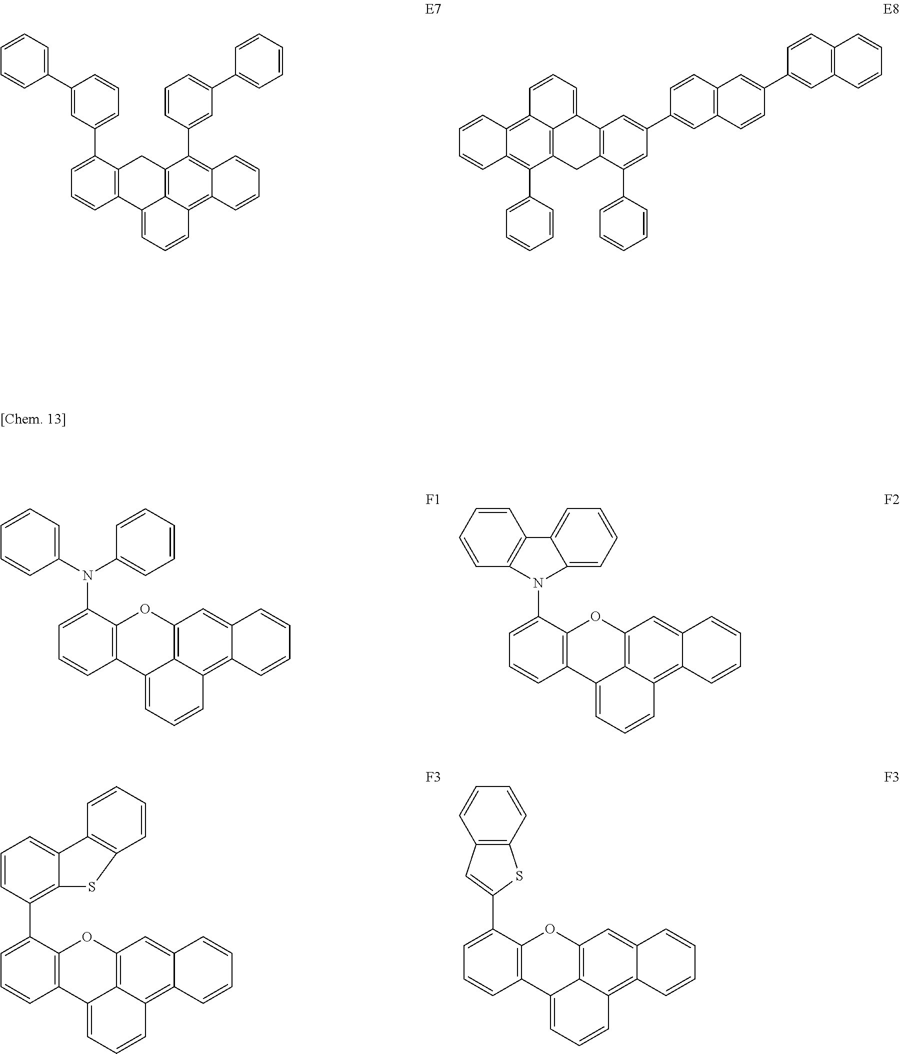

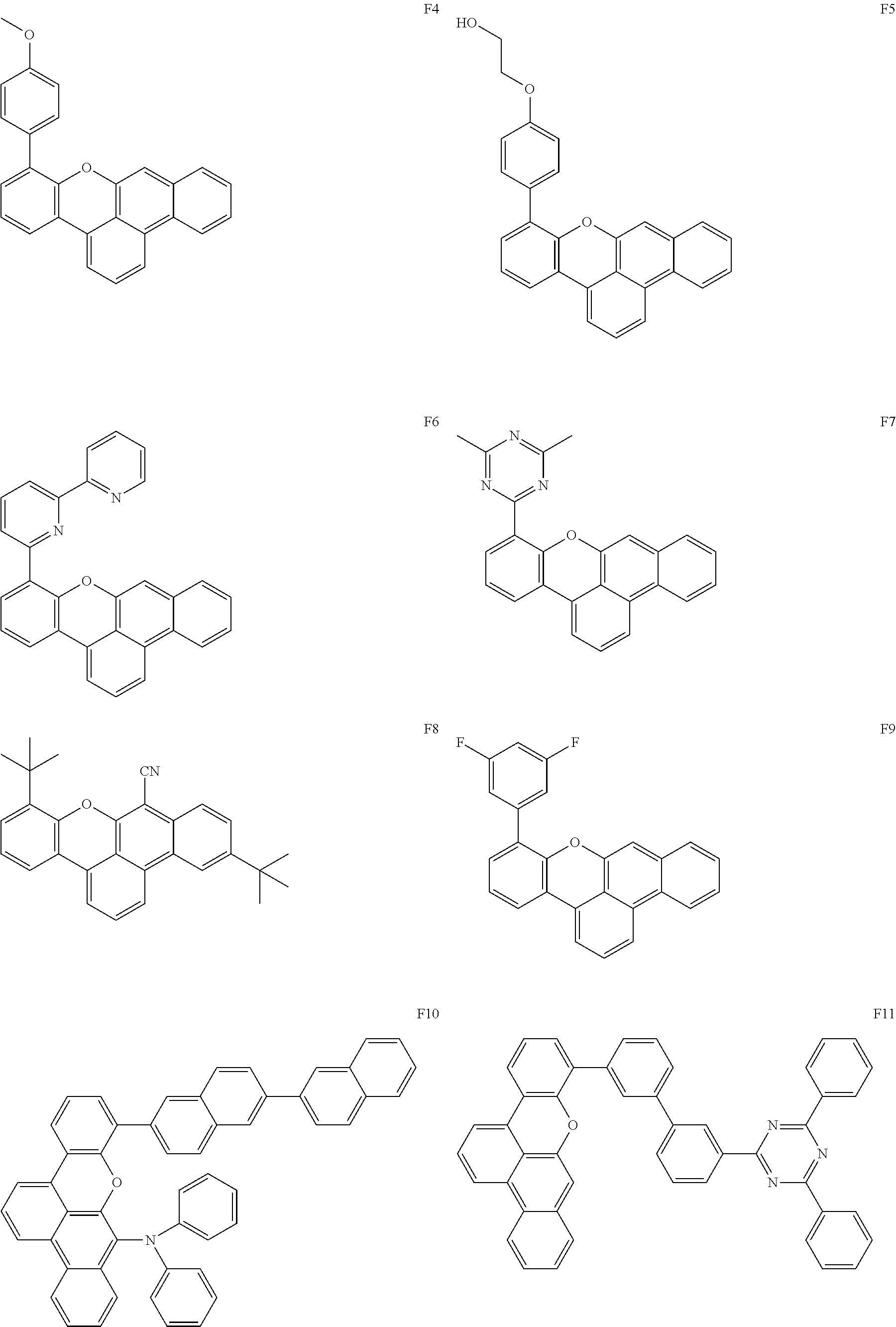

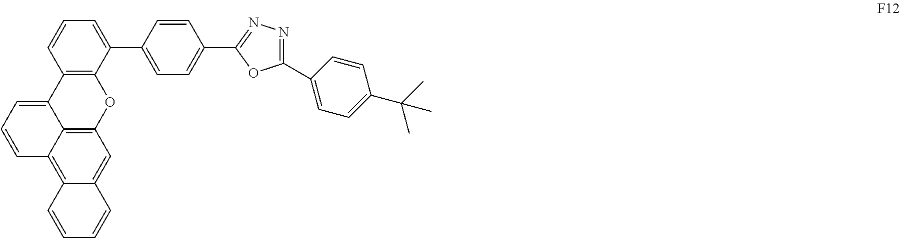 [SMILES: CC(C)(C)c1ccc(-c2nnc(-c3ccc(-c4cccc5c4Oc4cc6ccccc6c6cccc-5c46)cc3)o2)cc1]

### Properties of Exemplary Compounds

The exemplary dibenzoxanthene compounds according to this embodiment are divided into groups A to F.

The above exemplary compounds all have high T1 levels because of their dibenzoxanthene basic backbone. The exemplary compounds are grouped according to the positions of substituents. The effects characteristic of the substituted positions will now be described.

The compounds in group A have particularly high chemical stability and electrochemical stability and maintain high T1 levels after the substituent is attached.

Thus, if the compounds in group A are used for an organic phosphorescent device, particularly as a host material for a light-emitting layer, the device has a higher luminous efficiency and a longer life.

The exemplary compounds in group A are dibenzoxanthene compounds having an aryl group at R₁ and hydrogen at each of R₂ to R₇ in general formula [1]. The aryl group is selected from the group consisting of phenyl, naphthyl, biphenyl, fluorenyl, phenanthrenyl, chrysenyl, and picenyl. The aryl group is optionally substituted with at least one substituent selected from the group consisting of alkyl groups having 1 to 4 carbon atoms, phenyl, biphenyl, terphenyl, naphthyl, binaphthyl, and fluorenyl. The phenyl, biphenyl, terphenyl, naphthyl, binaphthyl, and fluorenyl substituents are optionally further substituted with at least one alkyl group having 1 to 4 carbon atoms.

The compounds in group B have a large dihedral angle between the plane of the dibenzoxanthene basic backbone and the plane of the aryl group because of steric hindrance between the aryl group and the adjacent hydrogen atom, as shown in the following structural formula.

That is, the substituent and the basic backbone are in a twisted conformation. This inhibits molecular stacking, thus providing better film-forming properties.

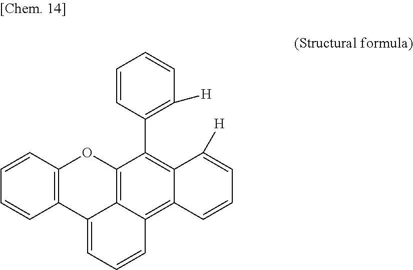 [SMILES: c1ccc(-c2c3c4c(cccc4c4ccccc24)-c2ccccc2O3)cc1]

The compounds in group C maintain high T1 levels after the substituent is introduced.

The compounds in group D have lower S1 levels because the substituent is positioned for extended conjugation.

The compounds in group E have substituents introduced at two or more positions. These compounds provide better film-forming properties because the positions adjacent to the oxygen atoms are protected and also because the multiple substituents alleviate the molecular rigidity. In particular, the compounds having substituents at R₁ and R₅ have high T1 levels and shallow HOMO levels.

The compounds in group F have a substituent containing a hetero atom. A larger change in HOMO level can be achieved by the electronic effect of the heteroatom.

The exemplary compounds in groups A to E are dibenzoxanthene compounds represented by general formula [1] where R₁ to R₇ are selected from the group consisting of hydrogen, substituted or unsubstituted alkyl groups, and substituted or unsubstituted aryl groups.

The exemplary compounds in groups A to E have high molecular stability because the substituent is composed only of a hydrocarbon.

### Description of Synthesis Route

An example of the synthesis route of an organic compound according to this embodiment will be described. The reaction formula is illustrated below.

Compounds G1 and G2 can be reacted by heating in, for example, dimethylformamide (DMF) under the presence of cesium carbonate and, as a catalyst, Pd(OAc)₂ and PPh₃ to synthesize intermediate G3.

Intermediate G3 can then be reacted with compound G4 in, for example, 1,4-dioxane under the presence of potassium acetate and, as a catalyst, Pd₂(dba)₃ and XPhos ligand to synthesize a dibenzoxanthene compound according to this embodiment.

Various compounds G1, G2, and G4 can be used to synthesize various dibenzoxanthene compounds. Examples of such compounds are shown in Tables 4-1 and 4-2. With the combinations shown in Tables 4-1 and 4-2, the exemplary compounds shown in Table 2 can be synthesized through the illustrated synthesis route.

To attach a substituent to the basic backbone, for example, dibenzoxanthene basic backbone b-1 can be reacted with sec-butyllithium in tetrahydrofuran (THF) at −78° C. to form lithio derivative b-2.

Lithio derivative b-2 can then be reacted with compound b-3 to form pinacolborate b-4 or with compound b-5 to form bromide b-6.

Alternatively, dibenzoxanthene backbone b-1 can be reacted with bromosuccinimide (NBS) in, for example, dichloromethane to form bromide b-7.

Thus, the exemplary compounds in groups A to D can be synthesized by the above synthesis methods, and the exemplary compounds in groups E and F can be synthesized by combining the above synthesis methods.

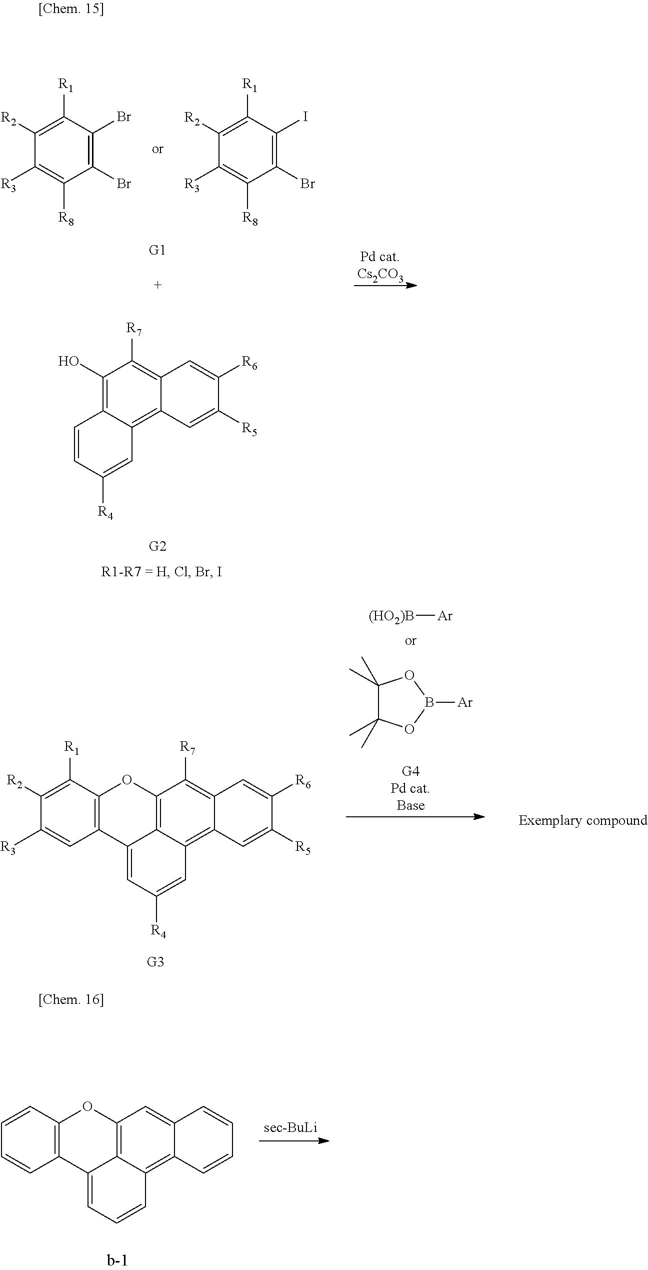

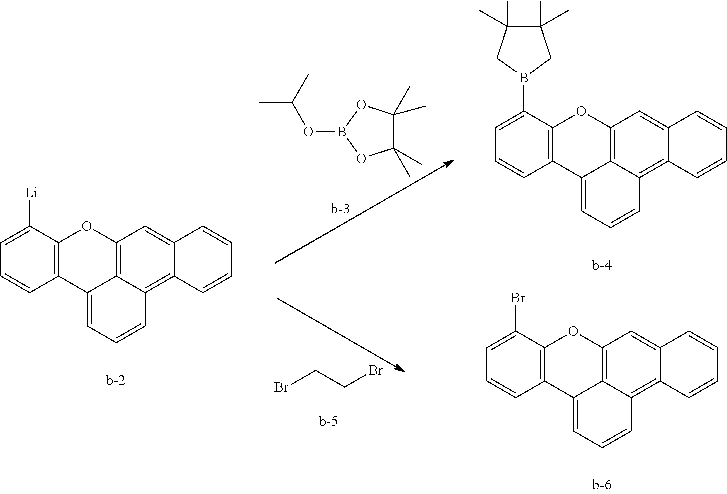

TABLE 4-1

| | | | | Exemplary |
|---|---|---|---|---|
| | | | | compound |
| | Compound G1 | Compound G2 | Compound G4 | No. |
| 1 | 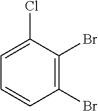 | 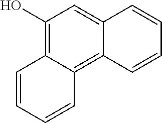 [SMILES: Oc1cc2ccccc2c2ccccc12] | 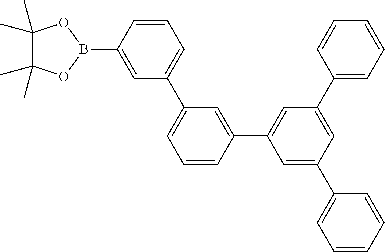 [SMILES: CC1(C)OB(c2cccc(-c3cccc(-c4cc(-c5ccccc5)cc(-c5ccccc5)c4)c3)c2)OC1(C)C] | A2 |
| 2 | 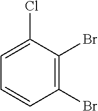 | 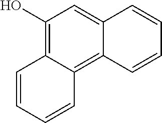 [SMILES: Oc1cc2ccccc2c2ccccc12] | 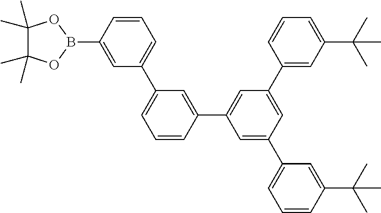 [SMILES: CC(C)(C)c1cccc(-c2cc(-c3cccc(-c4cccc(B5OC(C)(C)C(C)(C)O5)c4)c3)cc(-c3cccc(C(C)(C)C)c3)c2)c1] | A18 |
| 3 | 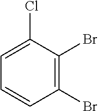 | 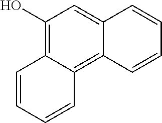 [SMILES: Oc1cc2ccccc2c2ccccc12] | 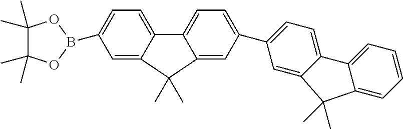 [SMILES: CC1(C)c2ccccc2-c2ccc(-c3ccc4c(c3)C(C)(C)c3cc(B5OC(C)(C)C(C)(C)O5)ccc3-4)cc21] | A19 |
| 4 | 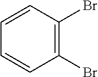 | 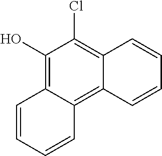 [SMILES: Oc1c(Cl)c2ccccc2c2ccccc12] | 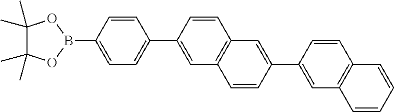 [SMILES: CC1(C)OB(c2ccc(-c3ccc4cc(-c5ccc6ccccc6c5)ccc4c3)cc2)OC1(C)C] | B2 |
| 5 | 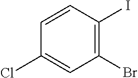 | 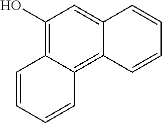 [SMILES: Oc1cc2ccccc2c2ccccc12] | 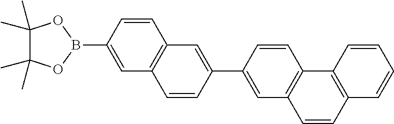 [SMILES: CC1(C)OB(c2ccc3cc(-c4ccc5c(ccc6ccccc65)c4)ccc3c2)OC1(C)C] | C4 |

TABLE 4-2

| | | | | |
|---|---|---|---|---|
| 6 | 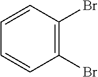 | 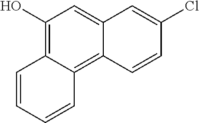 [SMILES: Oc1cc2cc(Cl)ccc2c2ccccc12] | 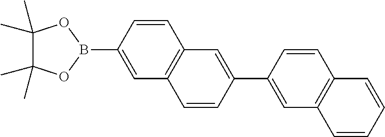 [SMILES: CC1(C)OB(c2ccc3cc(-c4ccc5ccccc5c4)ccc3c2)OC1(C)C] | C7 |
| 7 | 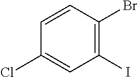 | 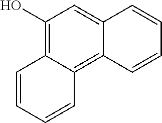 [SMILES: Oc1cc2ccccc2c2ccccc12] | 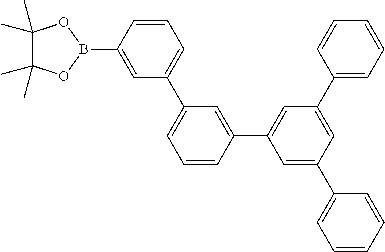 [SMILES: CC1(C)OB(c2cccc(-c3cccc(-c4cc(-c5ccccc5)cc(-c5ccccc5)c4)c3)c2)OC1(C)C] | D1 |
| 8 | 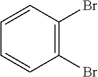 | 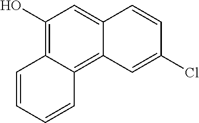 [SMILES: Oc1cc2ccc(Cl)cc2c2ccccc12] | 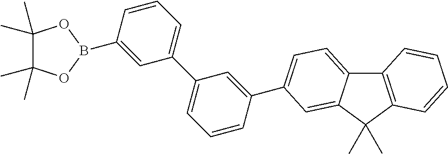 [SMILES: CC1(C)c2ccccc2-c2ccc(-c3cccc(-c4cccc(B5OC(C)(C)C(C)(C)O5)c4)c3)cc21] | D6 |
| 9 | 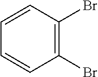 | 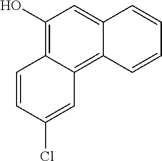 [SMILES: Oc1cc2ccccc2c2cc(Cl)ccc12] | 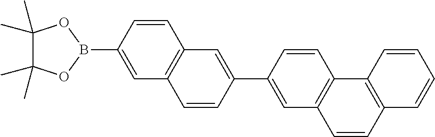 [SMILES: CC1(C)OB(c2ccc3cc(-c4ccc5c(ccc6ccccc65)c4)ccc3c2)OC1(C)C] | D12 |

### Description of Organic Light-Emitting Device According to Embodiment

Next, an organic light-emitting device according to an embodiment of the present invention will be described.

The organic light-emitting device according to this embodiment includes a pair of electrodes, i.e., an anode and a cathode, and an organic compound layer disposed therebetween. The organic compound layer contains an organic compound represented by formula [1].

The organic light-emitting device according to this embodiment can include either a single organic compound layer or a plurality of organic compound layers.

The organic compound layers are selected from the group consisting of hole-injecting layers, hole-transporting layers, light-emitting layers, hole-blocking layers, electron-transporting layers, electron-injecting layers, and exciton-blocking layers. It should be appreciated that a plurality of organic compound layers can be selected from the above group and can be used in combination.

The organic light-emitting device according to this embodiment is not limited to the above structure. For example, various layer structures can be selected, including a structure in which insulating layers are disposed between the electrodes and the organic compound layer, a structure including an adhesive layer or interference layer, and a structure including an electron-transporting layer or hole-transporting layer composed of two layers with different ionization potentials.

The organic light-emitting device according to this embodiment can have a top-emission structure, which outputs light from the electrode on the side facing the substrate, a bottom-emission structure, which outputs light from the side facing away from the substrate, or a structure that outputs light from both sides.

The organic light-emitting device according to this embodiment can include a light-emitting layer containing an organic compound according to an embodiment of the present invention.

The concentration of the host material in the light-emitting layer of the organic light-emitting device according to this embodiment is preferably 50% to 99.9% by weight, more preferably 80% to 99.5% by weight, of the entire light-emitting layer.

The concentration of the guest material relative to the host material in the light-emitting layer of the organic light-emitting device according to this embodiment is preferably 0.01% to 30% by weight, more preferably 0.1% to 20% by weight.

If a dibenzoxanthene compound according to an embodiment of the present invention is used as the guest material for the light-emitting layer of the organic light-emitting device, the concentration of the guest material relative to the host material is preferably 0.05% to 30% by mass, more preferably 0.1% to 10% by mass.

A dibenzoxanthene compound according to an embodiment of the present invention can be used as the host material or the guest material for the light-emitting layer.

In particular, if a dibenzoxanthene compound according to an embodiment of the present invention is used as a phosphorescent host material in combination with a guest material that emits light having an emission peak within the range of 550 to 680 nm, i.e., in the red region, the light-emitting device provides high luminous efficiency with low triplet energy loss.

The organic light-emitting device according to this embodiment can emit light in the range of 580 to 650 nm.

In this embodiment, the term “guest material” refers to a material that substantially determines the color of the light emitted by the organic light-emitting device and that itself emits light.

Examples of guest materials include, but not limited to, the following phosphorescent iridium complexes and platinum complex.

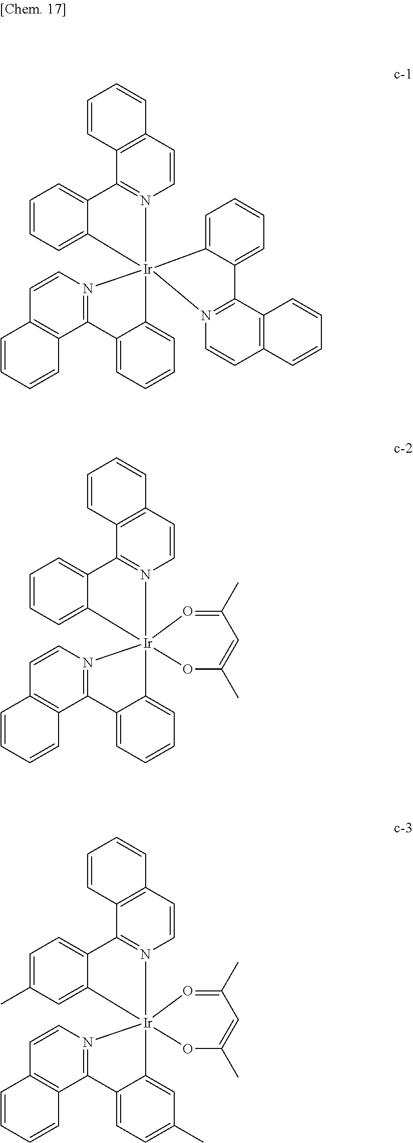

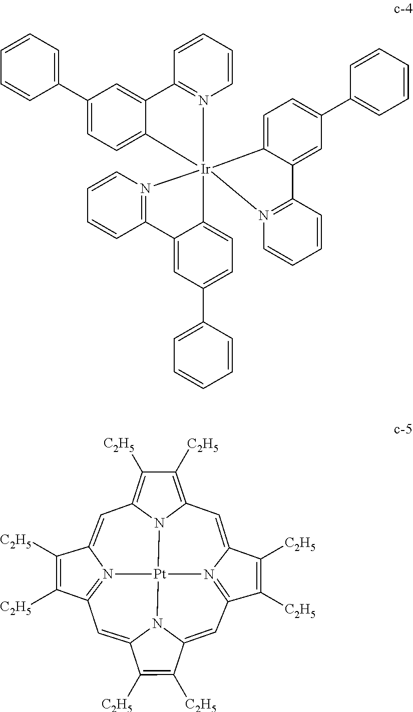

Fluorescent dopants can also be used. Examples of fluorescent dopants include fused ring compounds (e.g., fluorenes, naphthalenes, pyrenes, perylenes, tetracenes, anthracenes, and rubrene), quinacridones, coumarins, stilbenes, organoaluminum complexes such as tris(8-quinolinolato)aluminum, organoberyllium complexes, and polymers such as poly(phenylene vinylene)s, polyfluorenes, and polyphenylenes.

In particular, a compound having an anthracene backbone or a benzofluoranthene backbone can be used.

The term “compound having an anthracene backbone” refers to a compound having anthracene in the structure thereof and encompasses compounds having a substituted anthracene. The term “compound having a benzofluoranthene backbone” is similarly defined.

The organic light-emitting device according to this embodiment can emit light containing red phosphorescence because a dibenzoxanthene compound according to an embodiment of the present invention is suitable as a red phosphorescent host material.

The organic light-emitting device according to this embodiment can emit red light or a mixture of red light and light of other colors. For example, the organic light-emitting device can emit white light, which is a mixture of blue light, green light, and red light.

The organic light-emitting device according to this embodiment can include either a single light-emitting layer or a stack of light-emitting layers. For example, if the organic light-emitting device according to this embodiment is a white organic light-emitting device, the light-emitting layer structure thereof can be, but not limited to, any of the following structures:

(1) Single layer: a light-emitting layer containing blue, green, and red light-emitting materials

(2) Single layer: a light-emitting layer containing light blue and yellow light-emitting materials

(3) Two layers: a stack of a blue light-emitting layer and a light-emitting layer containing green and red light-emitting materials, or a stack of a red light-emitting layer and a light-emitting layer containing blue and green light-emitting materials

(4) Two layers: a stack of a light blue light-emitting layer and a yellow light-emitting layer

(5) Three layers: a stack of a blue light-emitting layer, a green light-emitting layer, and a red light-emitting layer

If the organic light-emitting device according to this embodiment is a white organic light-emitting device, it can include light-emitting layers that emit light other than red light, i.e., blue light and green light, which are mixed with red light to output white light. The light-emitting layer that emits red light can contain an organic compound according to an embodiment of the present invention.

The white organic light-emitting device according to this embodiment can be either a light-emitting device including a plurality of light-emitting layers or a light-emitting device including a light-emitting portion containing a plurality of light-emitting materials. If the white organic light-emitting device according to this embodiment is a light-emitting device including a plurality of light-emitting layers, at least one of the light-emitting layers contains a dibenzoxanthene compound according to an embodiment of the present invention. If the white organic light-emitting device according to this embodiment is a light-emitting device including a light-emitting portion containing a plurality of light-emitting materials, one of the light-emitting materials contained in the light-emitting portion is a dibenzoxanthene compound according to an embodiment of the present invention.

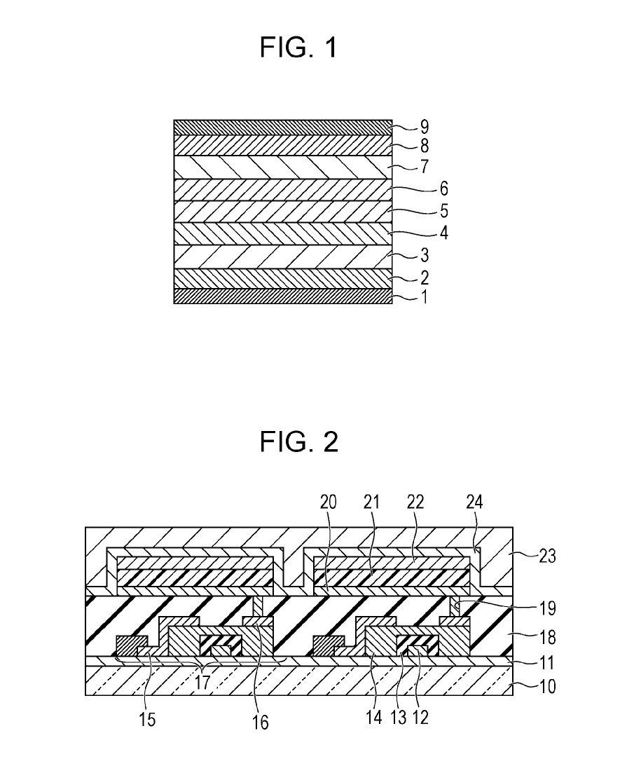

FIG. 1 is a schematic sectional view showing a device structure including a stack of light-emitting layers as an example of the white organic light-emitting device according to this embodiment. FIG. 1 illustrates an organic light-emitting device including three light-emitting layers that emit light of different colors. This structure will be described in detail below.

This organic light-emitting device includes an anode 1, a hole-injecting layer 2, a hole-transporting layer 3, a blue light-emitting layer 4, a green light-emitting layer 5, a red light-emitting layer 6, an electron-transporting layer 7, an electron-injecting layer 8, and a cathode 9 that are stacked on a substrate such as a glass substrate. The blue, green, and red light-emitting layers 4, 5, and 6, however, can be stacked in any other order.

The light-emitting layers 4, 5, and 6 are not necessarily stacked on top of each other, but can be arranged side-by-side. That is, the light-emitting layers 4, 5, and 6 can be arranged such that they are all in contact with the hole-transporting layer 3 and the electron-transporting layer 7.

Alternatively, the organic light-emitting device according to this embodiment can include a single light-emitting layer containing a plurality of light-emitting materials that emit light of different colors. In this case, the light-emitting materials can form their respective domains.

Examples of light-emitting materials used for the blue, green, and red light-emitting layers 4, 5, and 6 of the white organic light-emitting device according to this embodiment include, but not limited to, compounds having a chrysene backbone, compounds having a fluoranthene backbone, compounds having an anthracene backbone, boron complexes, and iridium complexes.

The white color in this embodiment is, for example, pure white or day white. The white color in this embodiment has a color temperature of, for example, 3,000 to 9,500 K. The CIE color coordinates of the light emitted by the white organic light-emitting device according to this embodiment are, for example, x=0.25 to 0.50 and y=0.30 to 0.42.

The organic light-emitting device according to this embodiment can optionally contain other known materials, including a hole-injecting material, a hole-transporting material, a host material, a guest material, an electron-injecting material, and an electron-transporting material. These materials can be either a low-molecular-weight material or a polymeric material.

These materials are illustrated below.

The hole-injecting material or the hole-transporting material can be a material having high hole mobility. Examples of low-molecular-weight and polymeric materials having hole-injecting properties or hole-transporting properties include, but not limited to, triarylamines, phenylenediamines, stilbenes, phthalocyanines, porphyrins, polyvinylcarbazoles, polythiophenes, and other conductive polymers.

The electron-injecting material or the electron-transporting material is selected taking into account the balance with the hole mobility of the hole-injecting material or the hole-transporting material.

Examples of materials having electron-injecting properties or electron-transporting properties include, but not limited to, oxadiazoles, oxazoles, pyrazines, triazoles, triazines, quinolines, quinoxalines, phenanthrolines, and organoaluminum complexes.

The anode material can be a material having a high work function. Example of such anode materials include elemental metals such as gold, platinum, silver, copper, nickel, palladium, cobalt, selenium, vanadium, and tungsten; alloys thereof; and metal oxides such as tin oxide, zinc oxide, indium oxide, indium tin oxide (ITO), and indium zinc oxide (IZO).

Conductive polymers such as polyaniline, polypyrrole, and polythiophene are also available. These electrode materials can be used alone or in combination. The anode 1 can be composed of either a single layer or a plurality of layers.

The cathode material can be a material having a low work function. Examples of such cathode materials include alkali metals such as lithium, alkaline earth metals such as calcium, and elemental metals such as aluminum, titanium, manganese, silver, lead, and chromium. Alloys of such elemental metals can also be used.

For example, magnesium-silver, aluminum-lithium, and aluminum-magnesium can be used. Also available are metal oxides such as ITO. These electrode materials can be used alone or in combination. The cathode 9 can be composed of either a single layer or a plurality of layers.

The individual layers of the organic light-emitting device according to this embodiment, including a layer containing an organic compound according to an embodiment of the present invention and layers containing other organic compounds, can be formed by a known process such as vacuum evaporation, ionization evaporation, sputtering, plasma deposition, and solution coating using a suitable solvent. Examples of coating processes include spin coating, dipping, casting, the Langmuir-Blodgett (LB) technique, and inkjet coating.

Evaporation or solution coating forms a layer that exhibits high stability with little or no crystallization over time. For coating, a film can be formed using a suitable binder resin.

Examples of binder resins include, but not limited to, polyvinylcarbazole resins, polycarbonate resins, polyester resins, acrylonitrile-butadiene-styrene (ABS) resins, acrylic resins, polyimide resins, phenolic resins, epoxy resins, silicone resins, and urea resins.

These binder resins can be used alone as a homopolymer or copolymer or as a mixture of two or more. Optionally, known additives such as plasticizers, antioxidants, and ultraviolet absorbers can be used.

### Applications of Organic Light-Emitting Device According to Embodiment

The organic light-emitting device according to this embodiment can be used as a component of a display or a lighting apparatus. Other applications include exposure light sources for electrophotographic image-forming apparatuses, backlights for liquid crystal displays, and white light sources combined with color filters. Examples of color filters include filters that transmit red, green, and blue light.

A display according to an embodiment of the present invention includes a display unit having a plurality of pixels, each including an organic light-emitting device according to an embodiment of the present invention.

Specifically, each pixel includes an organic light-emitting device according to an embodiment of the present invention and a transistor, as an example of an active device, configured to control the luminous intensity of the organic light-emitting device. The anode or the cathode of the organic light-emitting device is connected to a drain electrode or a source electrode of the transistor. The display can be used as an image display of a personal computer (PC).

Alternatively, the display can be configured as an image information processor including an input unit configured to receive image information from an area charge-coupled device (CCD) sensor, a linear CCD sensor, or a memory card and a display unit configured to display the input image.

The display unit of the image information processor can have a touch panel function. The touch panel function can be implemented in any manner.

Alternatively, the display can be used as a display unit of a multifunction printer.

A lighting apparatus according to an embodiment of the present invention is, for example, an indoor lighting apparatus. The lighting apparatus can emit, for example, white light, day white light, or light of any color in the blue to red region.

The lighting apparatus according to this embodiment includes an organic light-emitting device according to an embodiment of the present invention and an AC-DC converter circuit configured to supply a drive voltage to the organic light-emitting device. The lighting apparatus can include a color filter.

The AC-DC converter circuit used in this embodiment is a circuit configured to convert an alternating-current voltage to a direct-current voltage.

In this embodiment, the term “white” refers to a color with a color temperature of 4,200 K, and the term “day white” refers to a color with a color temperature of 5,000 K.

An image-forming apparatus according to an embodiment of the present invention includes a photoreceptor, a charging unit configured to charge a surface of the photoreceptor, an exposure unit configured to expose the photoreceptor to form an electrostatic latent image, and a developing unit configured to develop the electrostatic latent image formed on the surface of the photoreceptor. The exposure unit includes an organic light-emitting device according to an embodiment of the present invention.

Next, a display including organic light-emitting devices according to an embodiment of the present invention will be described with reference to FIG. 2.

FIG. 2 is a schematic sectional view of a display including organic light-emitting devices according to an embodiment of the present invention and thin-film transistors (TFTs), as an example of active devices, connected to the organic light-emitting devices.

This display includes a substrate 10 such as a glass substrate and a moisture-proof film 11 disposed on the substrate 10 to protect TFTs 17 and organic compound layers 21. Also provided on the substrate 10 are metal gates 12, gate insulators 13, and semiconductor layers 14.

The TFTs 17 each include a semiconductor layer 14, a drain electrode 15, and a source electrode 16. An insulating film 18 is disposed over the TFTs 17. The source electrodes 16 are connected through contact holes 19 to anodes 20 of the organic light-emitting devices.

The display according to this embodiment is not limited to the illustrated structure, but can have any structure in which the anodes 20 or the cathodes 22 are connected to source electrodes or drain electrodes of TFTs.

Although the organic compound layers 21 are shown as being a single layer in FIG. 2, they can be composed of a plurality of layers. A first protective layer 23 and a second protective layer 24 are disposed on the cathodes 22 to inhibit degradation of the organic light-emitting devices.

If the display according to this embodiment is a display that emits white light, the organic compound layers 21 in FIG. 2 are composed of a stack of light-emitting layers as illustrated in FIG. 1 so that the display emits white light.

The light-emitting layers of the display that emits white light according to this embodiment are not necessarily arranged as illustrated in FIG. 1; instead, light-emitting materials that emit light of different colors can be arranged side-by-side. Alternatively, a light-emitting layer containing light-emitting materials that emit light of different colors can be formed such that the light-emitting materials form domains in the light-emitting layer.

The TFTs 17 used as the active devices for the display according to this embodiment can be replaced by metal-insulator-metal (MIM) devices.

The TFTs 17 are not necessarily TFTs formed on a single-crystal silicon wafer, but can instead be TFTs including an active layer formed on an insulating surface of a substrate. Example of such TFTs include TFTs including an active layer formed of single-crystal silicon, TFTs including an active layer formed of a non-single-crystal silicon such as amorphous silicon or polycrystalline silicon, and TFTs including an active layer formed of a non-single-crystal oxide semiconductor such as IZO or indium gallium zinc oxide (IGZO).

The TFTs 17 provided for the organic light-emitting devices in this embodiment can be formed by directly processing a substrate such as a silicon substrate. That is, the TFTs 17 can be directly formed on a silicon substrate so as to share the same substrate with the organic light-emitting devices.

The type of active device of the display is selected depending on the definition of the display. For resolutions per inch of QVGA level, for example, active devices can be directly formed on a silicon substrate.

The display including the organic light-emitting devices according to this embodiment can stably display an image with high image quality after extended operation.

### EXAMPLES

Embodiments of the present invention are further illustrated by the following non-limiting examples.

### Example 1

### Synthesis of Exemplary Compound A7

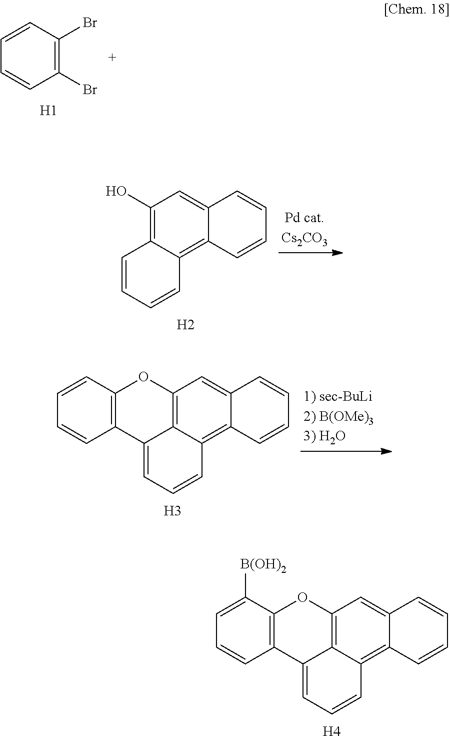

To 300 mL of DMF were added 865 mg (3.86 mmol) of palladium acetate and 4.04 g (15.4 mmol) of triphenylphosphine.

To the solution were added 7.29 g (30.9 mmol) of compound H1, 5.00 g (25.7 mmol) of compound H2, and 33.5 g (103 mmol) of cesium carbonate, and it was heated to 140° C. and was stirred for 7 hours.

After cooling, toluene was added, and it was filtered and concentrated. The residue was purified by silica gel column chromatography (mobile phase: heptane) to yield 4.14 g (yield: 60%) of a pale yellow solid of compound H3.

In 55 mL of THF was dissolved 1.0 g (3.7 mmol) of compound H3, and it was cooled to −78° C.

To the solution were added dropwise 0.84 mL (5.6 mmol) of N,N,N′,N′-tetramethylethane-1,2-diamine and 3.2 mL (4.5 mmol) of sec-butyllithium (1.4 mol/L), and it was stirred at −78° C. for 1 hour.

To the solution was added dropwise 1.3 mL (11 mmol) of trimethyl borate, and it was stirred for 2 hours while being heated to room temperature. Water was then added, and the reaction product was extracted with a mixture of toluene and THF and was dried over sodium sulfate.

Next, the solvent was distilled off, and the residue was washed with 100 mL of chloroform by dispersion. The solution was filtered and concentrated, and the residue was washed with n-heptane by dispersion. The solution was filtered, and the residue was dried to yield 436 mg (yield: 37%) of a pale yellow solid of compound H4.

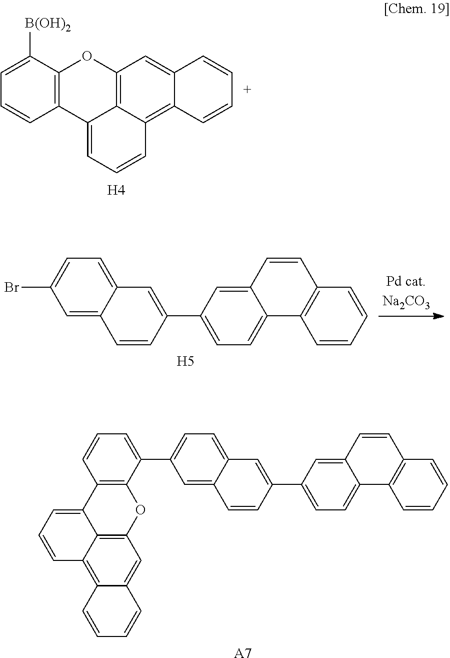

To a mixture of 10 mL of toluene, 5 mL of ethanol, and 5 mL of 10% by mass sodium carbonate aqueous solution were added 400 mg (1.28 mmol) of compound H4 and 442 mg (1.15 mmol) of compound H5.

To the solution was added 80 mg (0.07 mmol) of tetrakis(triphenylphosphine)palladium(0), and it was heated to 90° C. and was stirred for 2 hours.

After cooling, water and methanol were added, and it was filtered. The residue was dissolved in 600 mL of heated toluene at 130° C., followed by hot filtration. Hot filtration was performed using a Kiriyama funnel filled with silica gel.

The resulting filtrate was subjected to recrystallization from xylene to yield 426 mg (yield: 65%) of a pale yellow solid of compound A7.

Mass spectrometry showed M⁺=571, which corresponds to exemplary compound A7.

Proton nuclear magnetic resonance (¹H NMR) spectroscopy detected the structure of exemplary compound A7.

¹H NMR (THF, 500 MHz) δ (ppm): 8.95 (d, J=9.0 Hz, 1H), 8.87 (d, J=8.5 Hz, 1H), 8.64 (d, J=8.5 Hz, 1H), 8.63 (d, J=7.5 Hz, 1H), 8.47 (s, 1H), 8.44 (s, 1H), 8.24 (s, 1H), 8.23 (dd, J=9.0, 2.5 Hz, 1H), 8.17-8.11 (m, 5H), 7.98-7.93 (m, 3H), 7.86 (d, J=8.5 Hz, 1H), 7.79-7.75 (m, 2H), 7.72 (t, J=7.5 Hz, 1H), 7.64 (t, J=7.5 Hz, 1H), 7.58-7.48 (m, 3H), 7.32 (t, J=7.5 Hz, 1H), 7.16 (s, 1H).

The S1 level (optical bandgap) of exemplary compound A7 in a dilute toluene solution was measured and was found to be 420 nm.

The S1 level was determined as the absorption edge of a spectrum obtained by measuring the absorbance in the toluene solution (1×10⁻⁵ mol/L). The instrument used was a JASCO V-560 spectrophotometer.

The T1 level of exemplary compound A7 in a dilute toluene solution was measured and was found to be 533 nm.

The T1 level was determined as the rise in a spectrum obtained by cooling the toluene solution (1×10⁻⁴ mol/L) to 77 K and detecting phosphorescence at an excitation wavelength of 350 nm. The instrument used was a Hitachi F-4500.

### Example 2

### Synthesis of Exemplary Compound A8

Exemplary compound A8 was synthesized as in Example 1 except that compound H5 was replaced by compound H6 below.

Mass spectrometry showed M⁺=621, which corresponds to exemplary compound A8.

The S1 level of exemplary compound A8 in a dilute toluene solution was measured as in Example 1 and was found to be 420 nm.

The T1 level of exemplary compound A8 in a dilute toluene solution was also measured and was found to be 533 nm.

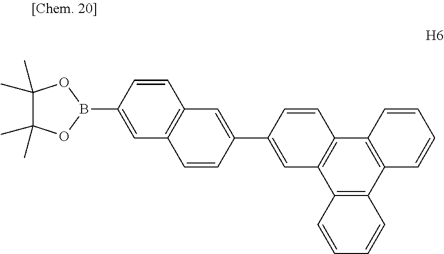 [SMILES: CC1(C)OB(c2ccc3cc(-c4ccc5c6ccccc6c6ccccc6c5c4)ccc3c2)OC1(C)C]

### Example 3

### Synthesis of Exemplary Compound A20

Exemplary compound A20 was synthesized as in Example 1 except that compound H5 was replaced by compound H7 below.

Mass spectrometry showed M⁺=587, which corresponds to exemplary compound A20.

The S1 level of exemplary compound A20 in a dilute toluene solution was measured as in Example 1 and was found to be 418 nm.

The T1 level of exemplary compound A20 in a dilute toluene solution was also measured and was found to be 530 nm.

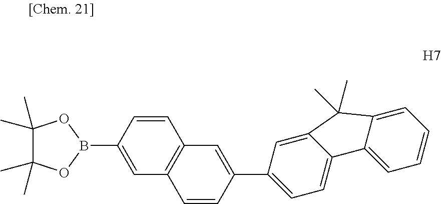 [SMILES: CC1(C)c2ccccc2-c2ccc(-c3ccc4cc(B5OC(C)(C)C(C)(C)O5)ccc4c3)cc21]

### Example 4

### Synthesis of Exemplary Compound B5

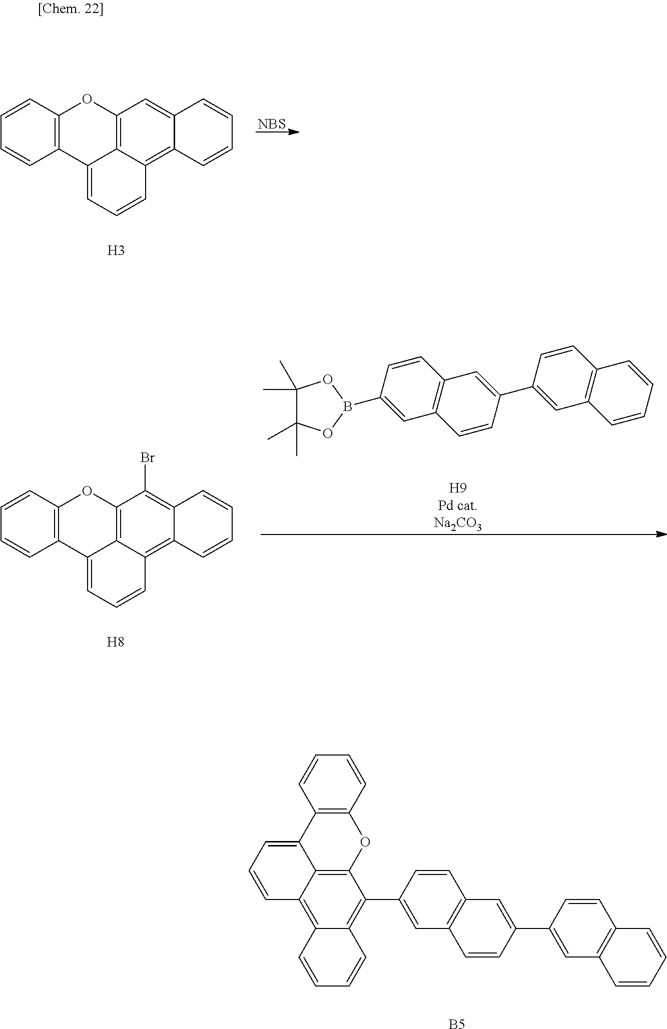

In 2 mL of dichloromethane was dissolved 50 mg (0.19 mmol) of compound H3. To the solution was added 33 mg (0.19 mmol) of NBS, and it was stirred at room temperature for 30 minutes.

Methanol was then added, and it was filtered. The residue was washed with water and methanol to yield 50 mg (yield: 77%) of a pale yellow green solid of compound H8.

To a mixture of 2 mL of toluene, 1 mL of ethanol, and 1 mL of 10% by mass sodium carbonate aqueous solution were added 50 mg (0.14 mmol) of compound H8 and 71 mg (0.16 mmol) of compound H9.

To the solution was added 10 mg (0.009 mmol) of tetrakis(triphenylphosphine)palladium(0), and it was heated to 90° C. and was stirred for 3 hours.

After cooling, water and methanol were added, and it was filtered. The residue was dissolved in heated toluene at 130° C., followed by hot filtration using a Kiriyama funnel filled with silica gel. The filtrate was subjected to recrystallization from xylene to yield 55 mg (yield: 75%) of a pale yellow solid of compound B5.

Mass spectrometry showed M⁺=587, which corresponds to exemplary compound B5.

The S1 level of exemplary compound B5 in a dilute toluene solution was measured as in Example 1 and was found to be 420 nm.

The T1 level of exemplary compound B5 in a dilute toluene solution was also measured and was found to be 540 nm.

### Example 5

### Synthesis of Exemplary Compound C2

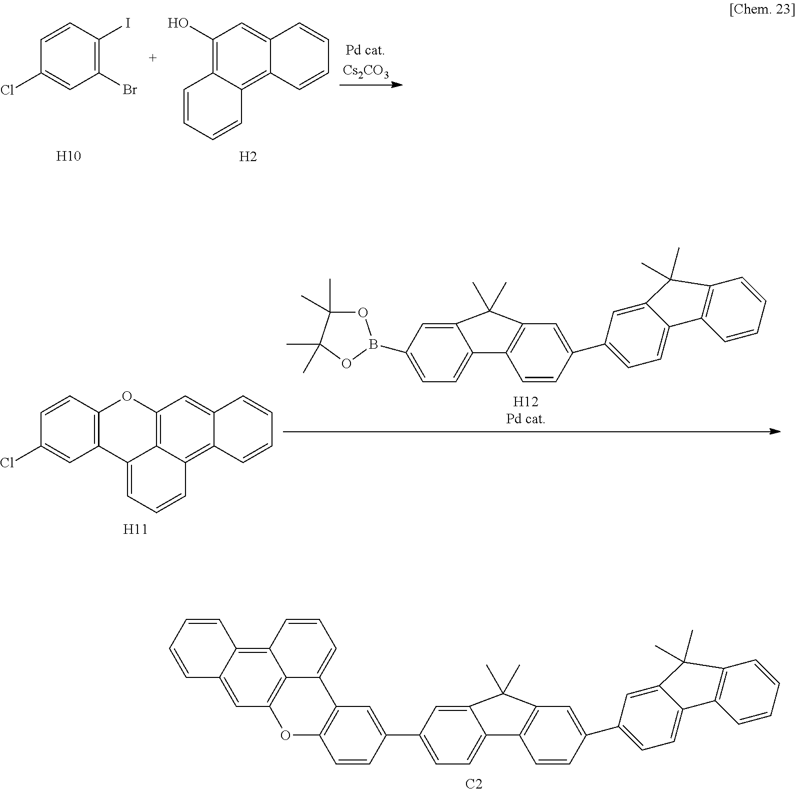

To 100 mL of DMF was added 1.19 g (1.03 mmol) of tetrakis(triphenylphosphine)palladium(0). To the solution were added 6.86 g (21.6 mmol) of compound H10, 4.00 g (20.6 mmol) of compound H2, and 26.8 g (82.4 mmol) of cesium carbonate, and it was heated to 140° C. and was stirred for 14 hours.

After cooling, toluene was added, and it was filtered and concentrated. The residue was purified by silica gel column chromatography (mobile phase: heptane/chloroform=20/1) to yield 342 mg (yield: 5%) of a pale yellow solid of compound C2.

To 5 mL of toluene were added 45 mg (0.050 mmol) of tris(dibenzylideneacetone)dipalladium(0) and 61 mg (0.15 mmol) of SPhos, and it was stirred at room temperature for 15 minutes.

To the solution were added 609 mg (1.19 mmol) of compound H12, 300 mg (0.99 mmol) of compound H11, 500 mg (2.38 mmol) of potassium phosphate, and 0.5 mL of water, and it was heated to 95° C. and was stirred for 7 hours.

After cooling, water and methanol were added, and it was filtered. The residue was dissolved in heated toluene at 130° C., followed by hot filtration (using a Kiriyama funnel filled with silica gel). The filtrate was subjected to recrystallization from toluene to yield 226 mg (yield: 35%) of a pale yellow solid of compound C2.

Mass spectrometry showed M⁺=653, which corresponds to exemplary compound C2.

The S1 level of exemplary compound C2 in a dilute toluene solution was measured as in Example 1 and was found to be 410 nm.

The T1 level of exemplary compound C2 in a dilute toluene solution was also measured and was found to be 530 nm.

### Example 6

### Synthesis of Exemplary Compound C3

Exemplary compound C3 was synthesized as in Example 5 except that compound H12 was replaced by compound H13 below.

Mass spectrometry showed M⁺=521, which corresponds to exemplary compound C3.

The S1 level of exemplary compound C3 in a dilute toluene solution was measured as in Example 1 and was found to be 408 nm.

The T1 level of exemplary compound C3 in a dilute toluene solution was also measured and was found to be 531 nm.

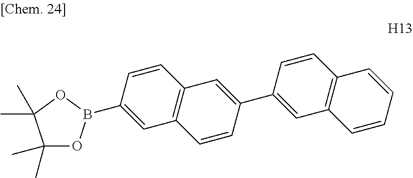 [SMILES: CC1(C)OB(c2ccc3cc(-c4ccc5ccccc5c4)ccc3c2)OC1(C)C]

### Example 7

In this example, an organic light-emitting device including, in order, a substrate, an anode, a hole-transporting layer, a light-emitting layer, an electron-transporting layer, and a cathode was fabricated by the following process.

An ITO film, serving as the anode, was deposited to a thickness of 120 nm on a glass substrate by sputtering to fabricate a transparent conductive support substrate (ITO substrate).

The following organic compound layers and electrode layers were continuously deposited on the ITO substrate by vacuum evaporation using resistance heating in a vacuum chamber at 10⁻⁵ Pa. The opposing electrodes were formed over an area of 3 mm².

Hole-injecting layer (40 nm): I1

Electron-blocking layer (10 nm): I2

Light-emitting layer (30 nm): host: A7, guest: c-1 (4% by weight)

Hole-blocking layer (10 nm): I3

Electron-transporting layer (50 nm): I4

First metal electrode layer (1 nm): LiF

Second metal electrode layer (100 nm): Al

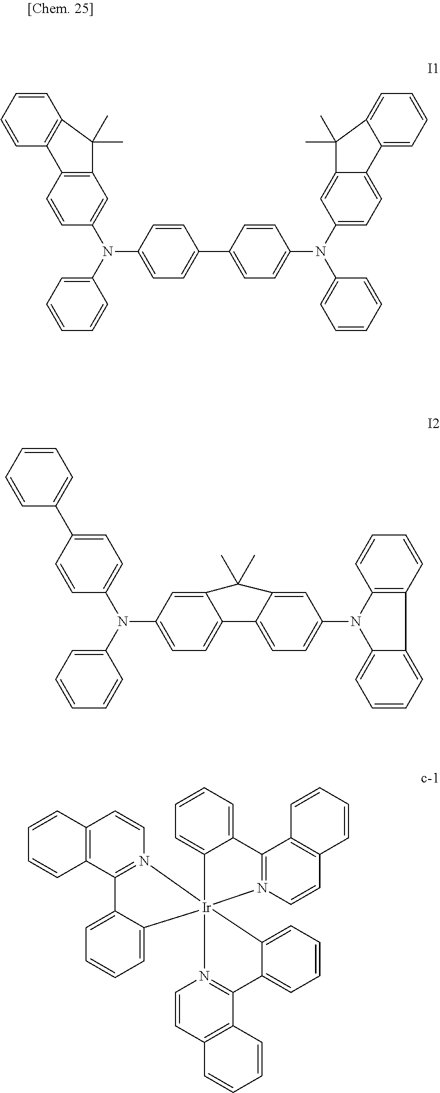

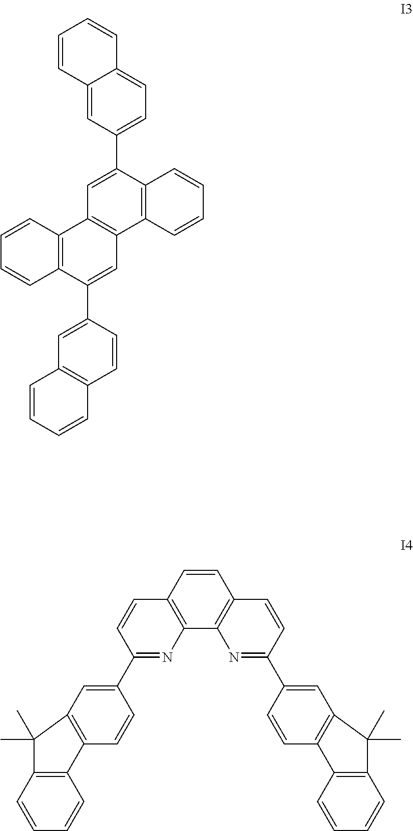

When a voltage of 4.0 V was applied to the resulting organic light-emitting device, with the ITO electrode being positive and the aluminum electrode being negative, red light was observed with a luminous efficiency of 11 cd/A.

The CIE color coordinates of the light emitted by the organic light-emitting device were (x, y)=(0.68, 0.32). After the organic light-emitting device operated at low voltage, i.e., 100 mA/cm², for 100 hours, the decrease in luminance was less than 10%.

### Example 8

An organic light-emitting device was fabricated as in Example 7 except that compound A7, used as the host material for the light-emitting layer, was replaced by exemplary compound A8.

When a voltage of 4.0 V was applied to the resulting organic light-emitting device, with the ITO electrode being positive and the aluminum electrode being negative, red light was observed with a luminous efficiency of 10.8 cd/A.

The CIE color coordinates of the light emitted by the organic light-emitting device were (x, y)=(0.68, 0.32). After the organic light-emitting device operated at low voltage, i.e., 100 mA/cm², for 100 hours, the decrease in luminance was less than 10%.

### Example 9

An organic light-emitting device was fabricated as in Example 7 except that compound A7, used as the host material for the light-emitting layer, was replaced by exemplary compound A20.

When a voltage of 3.9 V was applied to the resulting organic light-emitting device, with the ITO electrode being positive and the aluminum electrode being negative, red light was observed with a luminous efficiency of 11.1 cd/A.

The CIE color coordinates of the light emitted by the organic light-emitting device were (x, y)=(0.68, 0.32). After the organic light-emitting device operated at low voltage, i.e., 100 mA/cm², for 100 hours, the decrease in luminance was less than 10%.

### Example 10

An organic light-emitting device was fabricated as in Example 7 except that compound A7, used as the host material for the light-emitting layer, was replaced by exemplary compound C3.

When a voltage of 4.0 V was applied to the resulting organic light-emitting device, with the ITO electrode being positive and the aluminum electrode being negative, red light was observed with a luminous efficiency of 12.3 cd/A.

The CIE color coordinates of the light emitted by the organic light-emitting device were (x, y)=(0.68, 0.32). After the organic light-emitting device operated at low voltage, i.e., 100 mA/cm², for 100 hours, the decrease in luminance was less than 10%.

### Results and Discussion

As shown above, a dibenzoxanthene compound according to an embodiment of the present invention has a high T1 level, a narrow bandgap, and a shallow HOMO level, thus providing an organic light-emitting device that has high luminous efficiency, that operates at low voltage, and that has a long life.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-149154, filed Jul. 3, 2012, which is hereby incorporated by reference herein in its entirety.

According to an embodiment of the present invention, a novel dibenzoxanthene compound having a high T1 level and a narrow bandgap can be provided. The novel dibenzoxanthene compound can be used to provide an organic light-emitting device that has high luminous efficiency and that operates at low voltage.

### REFERENCE SIGNS LIST

4 blue light-emitting layer
5 green light-emitting layer
6 red light-emitting layer
17 TFT
20 anode
21 organic compound layer
22 cathode

1. A dibenzoxanthene compound represented by general formula [1]: 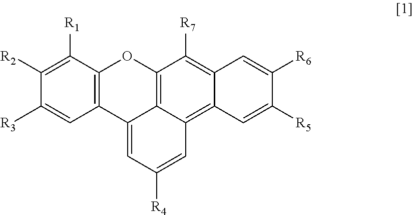
wherein R₁ to R₇ are each independently selected from the group consisting of hydrogen, substituted or unsubstituted alkyl groups, substituted or unsubstituted aryl groups, substituted or unsubstituted heterocyclic groups, substituted or unsubstituted aryloxy groups, substituted or unsubstituted alkoxy groups, substituted or unsubstituted amino groups, silyl groups, and cyano groups.

2. The dibenzoxanthene compound according to claim 1, wherein R₁ to R₇ are selected from the group consisting of hydrogen, substituted or unsubstituted alkyl groups, and substituted or unsubstituted aryl groups.

3. The dibenzoxanthene compound according to claim 1, wherein the dibenzoxanthene compound is represented by general formula [2]: 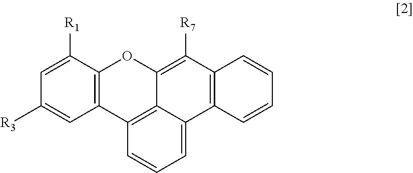
wherein R₁, R₃, and R₇ are each independently selected from the group consisting of hydrogen, substituted or unsubstituted alkyl groups, and substituted or unsubstituted aryl groups.

4. The dibenzoxanthene compound according to claim 1, wherein the dibenzoxanthene compound is represented by general formula [3]: 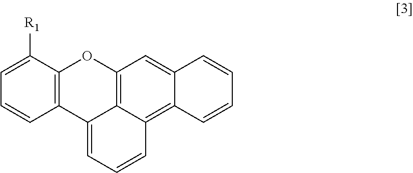
wherein:
R₁ is an aryl group, the aryl group being selected from the group consisting of phenyl, naphthyl, biphenyl, fluorenyl, phenanthrenyl, triphenylenyl, chrysenyl, and picenyl;
the aryl group is optionally substituted with at least one substituent selected from the group consisting of alkyl groups having 1 to 4 carbon atoms, phenyl, biphenyl, terphenyl, naphthyl, binaphthyl, fluorenyl, phenanthrenyl, triphenylenyl, chrysenyl, and picenyl; and
the phenyl, biphenyl, terphenyl, naphthyl, binaphthyl, fluorenyl, phenanthrenyl, triphenylenyl, chrysenyl, and picenyl substituents are optionally further substituted with at least one alkyl group having 1 to 4 carbon atoms.

5. The dibenzoxanthene compound according to claim 3, wherein:
the aryl group is selected from the group consisting of phenyl, biphenyl, naphthyl, fluorenyl, and phenanthrenyl; and
the aryl group is optionally substituted with at least one substituent selected from the group consisting of phenyl, biphenyl, naphthyl, fluorenyl, and phenanthrenyl.

6. An organic light-emitting device comprising:
a pair of electrodes; and
at least one organic compound layer disposed between the pair of electrodes,
wherein the at least one organic compound layer comprises the dibenzoxanthene compound according to claim 1.

7. The organic light-emitting device according to claim 6, wherein:
the at least one organic compound layer includes a light-emitting layer comprising a host material and a guest material; and
the host material is the dibenzoxanthene compound.

8. The organic light-emitting device according to claim 7, wherein the guest material is an iridium complex.

9. The organic light-emitting device according to claim 6, wherein:
the at least one organic compound layer includes a plurality of light-emitting layers;
at least one of the plurality of light-emitting layers comprises the dibenzoxanthene compound;
the plurality of light-emitting layers emit light of different colors; and
the organic light-emitting device emits white light.

10. A display comprising:
a plurality of pixels,
wherein at least one of the plurality of pixels comprises the organic light-emitting device according to claim 6 and an active device connected to the organic light-emitting device.

11. An image information processor comprising:
an input unit configured to receive image information; and
a display unit configured to display an image,
wherein the display unit is the display according to claim 10.

12. A lighting apparatus comprising:
the organic light-emitting device according to claim 6; and
an AC-DC converter circuit configured to supply a drive voltage to the organic light-emitting device.

13. An image-forming apparatus comprising:
a photoreceptor;
a charging unit configured to charge a surface of the photoreceptor;
an exposure unit configured to expose the photoreceptor to form an electrostatic latent image; and
a developing unit configured to develop the electrostatic latent image formed on the surface of the photoreceptor,
wherein the exposure unit comprises an organic light-emitting device according to claim 6.

14. An exposure unit for exposing a photoreceptor;
the exposure unit comprises the organic light-emitting device according to claim 6.
